(19) United States
(12) Patent Application Publication
(10) Pub. No.: US 2023/0110354 A1
(43) Pub. Date: Apr. 13, 2023

(54) THERAPEUTIC AGENT DELIVERY SYSTEMS AND METHODS OF FORMING AND USES THEREOF

(71) Applicant: The Regents of the University of Colorado, a Body Corporate, Denver, CO (US)

(72) Inventors: Jessica Anne Yu Rove, Aurora, CO (US); Dae Won Park, Englewood, CO (US); Adam J. Rocker, Broomfield, CO (US)

(21) Appl. No.: 18/075,627

(22) Filed: Dec. 6, 2022

Related U.S. Application Data
(63) Continuation of application No. PCT/US2021/037010, filed on Jun. 11, 2021.
(60) Provisional application No. 63/067,856, filed on Aug. 19, 2020.

(51) Int. Cl. A61K 31/167; A61K 9/51; A61L 29/14

(52) U.S. Cl. CPC A61K 31/167; A61K 9/5138; A61L 29/145

(57) ABSTRACT

A novel therapeutic agent delivery system, methods of use and methods of formation thereof are presented. The novel delivery system is comprised of novel nanoparticles capable of at least partially encapsulating a therapeutic agent such as an anesthetic, antimicrobial, growth factor or protein. The nanoparticles are embedded with in a crosslinked hydrogel. The hydrogel can be administered directly to a patient or may be coated onto a device such as a catheter. The delivery system allows for a sustained release of the therapeutic agent over an extended period of time.

### CROSS REFERENCE TO RELATED APPLICATIONS

This application is a continuation of International Patent Application No. PCT/US2021/037010 entitled “Therapeutic Agent Delivery Systems and Methods of Forming and Uses Thereof”, filed Jun. 11, 2021, which claims priority to U.S. Provisional Application No. 63/037,856 entitled “Therapeutic Agent Delivery Systems and Methods of Forming and Uses Thereof”, filed Jun. 11, 2020, the contents of which are hereby incorporated by reference into this disclosure.

### GOVERNMENT SUPPORT STATEMENT

This invention was made with Government support under Grant No. T32 HL072738 awarded by the National Institutes of Health (NIH). The Government has certain rights in the invention.

### FIELD OF INVENTION

This invention relates to therapeutic agent delivery systems. Specifically, the invention provides a novel biodegradable micelle-containing hydrogel system and methods of formation and use thereof.

### BACKGROUND OF THE INVENTION

Drug Delivery Systems

Both hydrogels and nanoparticles have been widely used for controlled drug release in patients, however one drawback includes the release of the drug too quickly. Many nanoparticle-hydrogel delivery systems have been developed, however not all delivery systems can be used to deliver all drugs or treat all diseases. Each delivery system is unique with respect to the composition of the hydrogel, crosslinking of the hydrogel, composition and type of nanoparticle to use with the hydrogel, types of therapeutic agents that can be encapsulated in the particular nanoparticle, stability of the composition, etc.

The inventors have developed a novel therapeutic agent delivery system which uses novel nanoparticles embedded within a crosslinked hydrogel to provide sustained release of a therapeutic agent. The use of the novel delivery system can be varied depending on the type of therapeutic agent used. For example, the inventors have expanded the use of the delivery system to deliver growth factors to aid in angiogenesis after myocardial infarction. In a different embodiment, the delivery system can be coated onto a catheter, such as a chest tube, to sustainably deliver an anesthetic agent. The shortcomings of the current state of the art regarding both uses is described below.

Treatment of Myocardial Infarction with Growth Factors

Coronary artery disease (CAD) is one of the leading causes of death in the United States, faulted for approximately 1 in 7 deaths with 660,000 new coronary attacks each year.¹ One consequence of CAD is a myocardial infarction (MI), which inhibits the flow of blood and vital nutrients to the heart. The current standard of care for MI aims for early reperfusion of the occluded vessels to prevent further cell death using surgical or pharmacological agents.² However, biomedical approaches to restore blood supply to ischemic myocardium, via highly localized angiogenesis, may present a faster and less invasive MI treatment option, with the essential benefit of inducing cardiac tissue regeneration.³ These therapies should aim to induce myocardial regeneration, reduce cardiomyocyte cell death, promote self-repair, attenuate harmful remodeling, and ultimately achieve long-term improvement in cardiac function.⁴ To this extent, a multifactorial protein delivery approach toward treating these various pathologies resulting from MI was designed.

An injectable, sulfonated reverse thermal gel (SRTG) for the controlled delivery of positively charged proteins to promote therapeutic angiogenesis has recently been developed.⁵ The SRTG is composed of poly(serinol hexamethylene urea) (PSHU) conjugated with poly(N-isopropylacrylamide) (PNIPAM) and sulfonate groups (SPSHU-PNIPAM or SRTG). The PSHU backbone is highly functionalizable and has been shown to exhibit a favorable microenvironment for neuronal⁶⁻⁹ and cardiac¹⁰⁻¹² tissue engineering applications. The negatively charged sulfonate groups control the release of positively charged proteins by utilizing their electrostatic binding interaction. The SRTG delivery system protects and steadily releases vascular endothelial growth factor (VEGF).^{5, 12} The delivery of VEGF from SRTG in a subcutaneous injection mouse model showed substantial vascularization properties by inducing vessel formation, recruitment and differentiation of vascular endothelial cells, and vessel stabilization by pericytes.⁵

Additionally, the intramyocardial injection of SRTG loaded with VEGF in a MI mouse model exhibited considerable cardioprotective and vascularization properties toward treating MI.¹² However, it has been shown that multiple growth factors need to be delivered in a sequential manner to produce a more stable and mature neovascular network.¹³⁻¹⁷ Platelet-derived growth factor (PDGF) is responsible for pericyte cell recruitment after initial luminal formation, which directs these cells to attach to neovessels and provide stabilization.¹⁸ Thus, PDGF is vital at a later phase of angiogenesis to prevent the formation of aberrant and leaky vessels and their eventual regression.¹⁹ When presented simultaneously, studies have shown that early-stage angiogenic factors can have conflicting effects on proteins involved in later stages and vice versa.^{16, 17, 20} Therefore, it is critical to mimic the physiological mechanism of angiogenesis by sequentially delivering multiple growth factors involved in different phases of this process.

Recent studies on developing therapies for cardiac repair have mainly focused on revascularization of damaged myocardium; however, it is vital to explore therapeutic treatments toward limiting the over-reactive and extended inflammation response following cardiac injury, to avoid the vicious cycle of MI to heart failure (HF).²¹ Interleukin-10 (IL-10) is a potent anti-inflammatory cytokine with broad immunoregulatory activity, and it can suppress many proinflammatory mediators involved in chronic HF.^{22, 23} It has been shown that IL-10 treatment in acute MI rodent models significantly improved left ventricular (LV) function, reduced infarct size, and attenuated infarct wall thinning.^{22, 24} Although these results are promising, IL-10 has a short half-life of less than 5 h in vivo, and it normally requires repeated injections to reach therapeutic efficacy, potentially causing undesirable side effects with a high treatment cost.²⁵ Human IL-10 has been shown to have strong heparin-binding affinity properties (Kd=54±7 nM).²⁶ The SRTG delivery system may provide a localized and sustained release of IL-10 through electrostatic interactions, which could reduce issues associated with high therapeutic doses.

Treatment of Chest Tube Pain

In 2000, the Joint Commission recommended making pain the 5th vital sign, increasing the focus on postoperative pain control.⁵⁵ In the subsequent two decades, prescription opioid medications have contributed significantly to what is now recognized as an opioid crisis and a Public Health Emergency as declared by the United States Department of Health and Human Services.^{56, 57} In 2017 there were 131 opioid overdose deaths a day, and approximately 1.7 million people aged 12 and older reported a prescription pain reliever use disorder in the past year.⁵⁷ In a multi-institutional analysis of major elective operations, cardiothoracic surgery operations were specifically identified as predictors of in-hospital and post-operative opioid overdose including coronary artery bypass grafting (CABG), pneumonectomy and lobectomy.⁵⁸

Chest tubes are a necessary part of performing intrathoracic operations with the vital function of draining blood and preventing tamponade, evacuating air and preventing pneumothoraces. With over 500 open heart cases performed a year at University of Colorado Hospital (UCH), and an average of 2-3 drains per case, over 1,000 chest drains are placed annually for cardiac operations alone. Chest tubes account for a large proportion of pain stimulation after a cardiac or thoracic operation. In studies examining pain and number of chest tubes, fewer chest tubes reduces pain and opioid use after thoracotomy.⁵⁹ Numerous studies have examined techniques to minimize pain and opioid use after chest incisions including epidurals, peri-vertebral catheters, rib blocks, pleural infusions of bupivacaine and application of lidocaine jelly to chest tubes.⁶⁰⁻⁶² There are a variety of reasons why none of these techniques have been widely adopted: inconsistent results, undesirable hemodynamic side effects, too costly, specialized equipment, required teaching for staff, or significant modification of intraoperative practice. A consistent finding across these studies is that opioid use is reduced when pain from the chest tube is reduced.

In light of the shortcomings of the prior art, what is needed is a novel, non-opioid analgesic adjunct to reduce pain associated with postoperative chest tubes.

### SUMMARY OF INVENTION

The inventors have developed a novel therapeutic agent delivery system that can be used to deliver one or more therapeutic agents to a patient. The novel delivery system is comprised of at least one nanoparticle at least partially encapsulating at least one therapeutic agent. The at least one nanoparticle is embedded within a crosslinked hydrogel. Administration of the delivery system to a patient allows for the therapeutic agent to be sustainably released in the patient. The therapeutic agents that can be used with the novel delivery system include anesthetics, antimicrobials, and growth factors. Depending on the type of therapeutic agent administered, this novel delivery system can be used to treat pain, infection, or even promote angiogenesis and reduce inflammation after myocardial infarction as described below.

Determining ideal strategies for the minimization of myocardial necrosis and optimization of cardiac repair following MI is one of the most important therapeutic targets of modern cardiology.^{27, 28} To address this therapeutic strategy, in an embodiment, a sequential protein delivery system comprising of SRTG encapsulating growth factor-loaded polymeric micelle nanoparticles (NPs) has been developed. VEGF and IL-10 were mixed into the aqueous polymer solution before gelling; these proteins should be released according to their binding affinity for sulfonate groups. Novel polymeric micelles were incorporated to provide the sequential and sustained release of PDGF. It was hypothesized that this system will deliver VEGF first, followed by IL-10, and finally PDGF, which may promote optimal cardiac repair after MI. By quickly initiating the angiogenesis process with VEGF, ischemic myocardium may be protected from necrosis. The subsequent delivery of IL-10 may repress proinflammatory mediators, which protects the heart from excessive inflammatory injury, while still allowing the important mechanism of clearing dead cells from the infarcted area.²⁹ The final delivery of PDGF from the encapsulated micelles may stabilize new vessels with pericytes and reduce the incidence of their regression. This specific combination of proteins for therapeutic delivery, targeting the different stages of pathological remodeling following MI, has not been previously utilized in a potential application for cardiac repair. In Example 3, the inventors evaluated the efficacy of this SRTG micelle delivery system for the spatiotemporal and sequential delivery of VEGF, IL-10, and PDGF for the promotion of mature angiogenic vessels and reducing inflammation.

In an embodiment, an anesthetic therapeutic agent, such as lidocaine, may be encapsulated within the nanoparticles which are embedded within the crosslinked hydrogel to form the delivery system. The hydrogel may optionally also contain an anesthetic, such as lidocaine. This delivery system may then be administered to the patient by various means including subcutaneous injection or topical application to relieve pain with sustained release of the lidocaine. In another embodiment, the novel delivery system can be stably coated onto a catheter, such as a chest tube, to relieve pain associated with placement of a chest tube as described herein.

In an embodiment, a system for delivery of a therapeutic agent is presented comprising: at least one nanoparticle wherein the at least one nanoparticle is a micelle formed of a polymer comprising a hydrophobic segment and at least one hydrophilic segment; a therapeutic agent encapsulated within the at least one nanoparticle; and a hydrogel wherein the at least one nanoparticle is embedded within the hydrogel. The system allows for sustained release of the therapeutic agent over an extended period of time. In some embodiments, the system further comprises an additional amount of the therapeutic agent being embedded within the hydrogel itself to allow for immediate release of the therapeutic agent in addition to the sustained release of the therapeutic agent from the nanoparticles.

The hydrophobic segment may be poly(serinol hexamethylene urea) (PSHU) and the at least one hydrophilic segment may be polyethylene glycol (PEG), polycaprolactone (PCL), poly(glycolic acid) (PGA), poly(lactic acid) (PLA), or poly(lactic acid-co-glycolic acid) (PLGA). The hydrogel may be gelatin crosslinked with glutaraldehyde.

The therapeutic agent may be selected from anesthetics, antimicrobials, or growth factors. In an embodiment, the therapeutic agent is an anesthetic selected from the group consisting of lidocaine, marcaine, bupivacaine, prilocaine, mepivacaine, etidocaine, ropivacaine, and levobupivacaine. In a different embodiment, the therapeutic agent is at least one growth factor selected from the group consisting of vascular endothelial growth factor (VEGF), platelet-derived growth factor (PDGF) and interleukin-10 (IL-10).

In a further embodiment, a method of reducing pain associated with placement of a catheter in a patient in need thereof is presented comprising the steps of: applying an anesthetic-eluting drug delivery system to a surface of a catheter to produce a coated catheter and inserting the coated catheter into the patient. The system allows for sustained release of the anesthetic over an extended period of time to relieve the pain associated with the placement of the catheter.

The drug delivery system coated onto the catheter comprises: at least one nanoparticle wherein the at least one nanoparticle is a micelle formed of a polymer comprising a hydrophobic segment, such as poly(serinol hexamethylene urea) (PSHU), and at least one hydrophilic segment, such as polyethylene glycol (PEG), polycaprolactone (PCL), poly(glycolic acid) (PGA), poly(lactic acid) (PLA), or poly(lactic acid-co-glycolic acid) (PLGA); an anesthetic encapsulated within the at least one nanoparticle; and a hydrogel wherein the at least one nanoparticle is embedded within the hydrogel which may be gelatin crosslinked with glutaraldehyde. The drug delivery system may further comprise an amount of the anesthetic being embedded in the hydrogel itself to allow for immediate release in addition to the sustained release of the anesthetic from the nanoparticles.

The anesthetic may be selected from the group consisting of lidocaine, marcaine, bupivacaine, prilocaine, mepivacaine, etidocaine, ropivacaine, and levobupivacaine.

In another embodiment, a method of promoting angiogenesis in patient after myocardial infarction is presented comprising: administering a therapeutic agent delivery system to the patient. The therapeutic agent delivery system comprises: at least one nanoparticle; a therapeutically effective amount of growth factors selected from vascular endothelial growth factor (VEGF), platelet-derived growth factor (PDGF), interleukin-10 (IL-10), or combinations thereof wherein at least one of the growth factors is at least partially encapsulated within the at least one nanoparticle; and a hydrogel wherein the at least one nanoparticle is embedded within the hydrogel. The growth factors not encapsulated within the nanoparticle are embedded in the hydrogel. The delivery system allows for both immediate and sustained release of the combination of growth factors.

In an embodiment, the PDGF is at least partially encapsulated within the at least one nanoparticle. The combination of growth factors are released sequentially with the VEGF released first, the IL-10 released second and the PDGF released last.

### BRIEF DESCRIPTION OF THE DRAWINGS

For a fuller understanding of the invention, reference should be made to the following detailed description, taken in connection with the accompanying drawings, in which:

FIG. 1A-B are a series of images depicting synthesis of the micelles. (A) Synthesis of PEG-PSHU-PEG by Step 1: synthesis of PSHU; Step 2: conjugation of PEG to terminal isocyanate groups on PSHU. (B) 1H NMR (500 MHz, DMSO-d6) spectrum of PEG-PSHU-PEG.

FIG. 2A-E are a series of images of the SRTG and the micelle NPs. (A-D) SEM images of the SRTG and micelle NPs. (A) 5% (w/v) of SRTG cross-section showing polymer sheets (scale bar=10 m). (B) Micelle NPs confirming the spherical structure (scale bar=2 m). (C) SRTG encapsulating micelle cross-section showing a porous configuration (scale bar=20 m). (D) Enlargement of SRTG encapsulating micelles with black arrows indicating micelles (scale bar=1 m). (E) Size distribution of micelles from DLS measurements.

FIG. 3 is a graph depicting Cumulative release profile of VEGF, IL-10, and PDGF from the delivery system in vitro. A burst release was observed for VEGF and IL-10 in the first 24 h, with a sustained release following over time. PDGF displayed minimal burst release and slowly released over 28 days, demonstrating the sequential release of all three factors from the SRTG micelle system. Error bars represent standard deviation.

FIG. 4A-E are a series of images depicting immunohistochemical assessment of vascularization by vessel formation, endothelial cell count, and functional vascular endothelial cell count. (A) Representative images show co-staining of CD31 (green), VWF (red), and DAPI (blue) 21 days after subcutaneous injections. Functional vascular cells were characteristic of CD31+ and VWF+. The scale bar represents 100 μm. Quantification of immunohistochemical assessment of vascularization by (B) endothelial cell count, (C) functional vascular endothelial cell count, and (D) ratio of functional vascular endothelial cells to total endothelial cells. (E) Represents the legend for the graphs. Error bars represent standard deviation and * indicates p<0.05.

FIG. 5A-D are a series of images depicting immunohistochemical assessment of mature vascularization by pericyte cell count. (A) Representative images show co-staining of CD31 (green), α-SMA (red), and DAPI (blue) 21 days after subcutaneous injections. Pericytes were characteristic of α-SMA+ associated with CD31+. The scale bar represents 100 μm. Quantification of immunohistochemical assessment of vascularization by (B) pericyte cell count and (C) ratio of pericytes to endothelial cells. (D) Represents the legend for the graphs. Error bars represent standard deviation and * indicates p<0.05.

FIG. 6A-C are a series of images depicting Immunohistochemical assessment of inflammatory response by macrophage cell count. (A) Representative images show staining of CD68 (red) and DAPI (blue) 7 and 21 days after injection. Scale bar represents 50 μm. (B) Quantification of immunohistochemical assessment of inflammatory response by macrophage cell count. (C) Represents the legend for the graphs. Error bars represent standard deviation and * indicates p<0.05.

FIG. 7 is a graph depicting a modified cumulative release profile of VEGF, IL-10 and PDGF from the delivery system in vitro. This graph was created by removing the burst release from the original release profile (FIG. 3) and starting the release profile for each factor with the data from day 1, while normalizing each release profile to start at 0. This further demonstrates the sequential release of all three factors from the SRTG micelle system since it can be difficult to discern the release rates from the full graph (1% cumulative release=5 ng of protein). Error bars represent standard deviation.

FIG. 8 is an image depicting the delivery system using growth factors and micelles in sulfonated reverse thermal gel (SRTG).

FIG. 9 is an SEM image of the lidocaine loaded micelles.

FIG. 10 is a graph depicting in vitro release profiles of different nanoparticle (NP) formulations loaded with 100 mg of lidocaine. The release profiles from the various NP formulations show the differences in lidocaine release rates are dependent on the amount of NP loaded into the hydrogels. The data display the sustained release of the lidocaine over 10 days for the 20 mg and 10 mg NP groups. The data for last day of each group shows last timepoint where lidocaine could be detected in the release solution.

FIG. 11 is a graph depicting in vitro release profiles of different nanoparticle formulations loaded with varied amounts of lidocaine. The release profiles from the various NP formulations show the differences in lidocaine release rates are dependent on the amount of NP and lidocaine loaded into the hydrogels. The data display the sustained release of the lidocaine over 11 days for the 50 mg lidocaine groups. The data for last day of each group shows last timepoint where lidocaine could be detected in the release solution.

FIG. 12 is a graph depicting in vitro release profiles of the nanoparticles loaded with varied amounts of lidocaine and embedded in gelatin hydrogels. The data display the sustained release of the lidocaine over eleven days. Error bars represent standard deviation.

FIG. 13A-B are a series of images depicting subcutaneous implantation mouse model of catheter tubing with and without gelatin-nanoparticle hydrogel coating. Representative photographs of harvested tissue showing fibrous capsule formation around tubing with and without hydrogel coating 14 days after implantation procedure. The uncoated tubing sample (A) shows similar fibrous tissue formation compared to coated tubing sample (B). This biological tissue response is due to a foreign body reaction from the implanted tubing.

FIG. 14 is a graph depicting the lidocaine release profile from the gelatin-lidocaine hydrogel comparing gel mold to coated tubing. Error bars represent standard deviation.

FIG. 15 is a graph depicting in vitro release profiles of lidocaine from different gelatin hydrogel formulations. The release profiles from the various gelatin formulations show the differences in lidocaine release rates are dependent on the concentration of gelatin and glutaraldehyde (GA). The data display the sustained release of the lidocaine over 10 days.

### DETAILED DESCRIPTION OF THE PREFERRED EMBODIMENT

In the following detailed description of the preferred embodiments, reference is made to the accompanying drawings, which form a part hereof, and within which are shown by way of illustration specific embodiments by which the invention may be practiced. It is to be understood that other embodiments may be utilized, and structural changes may be made without departing from the scope of the invention. The following description is not intended to limit the scope of the present description disclosed herein.

### Definitions

All numerical designations, including ranges, are approximations which are varied up or down by increments of 1.0 or 0.1, as appropriate. It is to be understood, even if it is not always explicitly stated that all numerical designations are preceded by the term “about”. It is also to be understood, even if it is not always explicitly stated, that the reagents described herein are merely exemplary and that equivalents of such are known in the art and can be substituted for the reagents explicitly stated herein.

The term “about” or “approximately” as used herein refers to being within an acceptable error range for the particular value as determined by one of ordinary skill in the art, which will depend in part on how the value is measured or determined. As used herein, the term “about” refers to ±10%.

Concentrations, amounts, solubilities, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include the individual values and sub-ranges within the indicated range. Thus, included in this numerical range are individual values such as 2, 3, and 4 and sub-ranges such as from 1-3, from 2-4 and from 3-5, etc. This same principle applies to ranges reciting only one numerical value. Furthermore, such an interpretation should apply regardless of the range or the characteristics being described.

As used in the specification and claims, the singular form “a”, “an” and “the” include plural references unless the context clearly dictates otherwise. For example, the term “a nanoparticle” includes a plurality of nanoparticles, including mixtures thereof.

As used herein, the term “comprising” is intended to mean that the products, compositions and methods include the referenced components or steps, but not excluding others. “Consisting essentially of” when used to define products, compositions and methods, shall mean excluding other components or steps of any essential significance. “Consisting of” shall mean excluding more than trace elements of other components or steps.

“Patient” is used to describe an animal, preferably a human, to whom treatment is administered, including prophylactic treatment with the compositions of the present invention.

“Pharmaceutically acceptable carrier” means any of the standard pharmaceutically acceptable carriers. The pharmaceutically acceptable carrier can include diluents, adjuvants, and vehicles, as well as implant carriers, and inert, non-toxic solid or liquid fillers, diluents, or encapsulating material that does not react with the active ingredients of the invention. Examples include, but are not limited to, phosphate buffered saline, physiological saline, water, and emulsions, such as oil/water emulsions. The carrier can be a solvent or dispersing medium containing, for example, ethanol, polyol (for example, glycerol, propylene glycol, liquid polyethylene glycol, and the like), suitable mixtures thereof, and vegetable oils. Formulations are described in a number of sources that are well known and readily available to those skilled in the art. For example, Remington's Pharmaceutical Sciences (Martin E W [1995] Easton Pa., Mack Publishing Company, 19^{th} ed.) describes formulations which can be used in connection with the subject invention. In some embodiments, phosphate buffered saline is used as the pharmaceutically acceptable carrier.

The terms “administer” or “administering” as used herein are defined as the process by which the compositions of the present invention are delivered to the patient for treatment or prevention purposes. The composition can be delivered via subcutaneous injection or topically in dosage unit formulations containing conventional nontoxic pharmaceutically acceptable carriers, adjuvants, and vehicles as desired. Administration may occur once or multiple times.

“Sustained release” as used herein refers to a composition comprising a therapeutically effective amount of a composition, when administered to a patient, continuously releases a stream of one or more therapeutic agents over a predetermined time period at a level sufficient to achieve a desired effect, such as preventing or treating infections; preventing or treating pain; or treating myocardial infarction by improving cardiac function, promoting angiogenesis or reducing inflammation, throughout the predetermined time period. Reference to a continuous release stream is intended to encompass release that occurs as the result of biodegradation of the composition, or component thereof, or as the result of metabolic transformation or dissolution of the added nutrients or other desired agents.

“Therapeutic agent” as used herein is defined as a substance, component or agent that has measurable specified or selective physiological activity when administered to an individual in a therapeutically effective amount. Examples of therapeutic agents as used in the present invention include growth factors, anesthetics and antimicrobials such as antibiotics, antivirals, antifungals, antiprotozoals, and antiparasitics. At least one therapeutic agent is used in the compositions of the present invention. In some embodiments, multiple therapeutic agents are used and are released in a sequential manner.

A “therapeutically effective amount” as used herein is defined as concentrations or amounts of components which are sufficient to effect beneficial or desired clinical results, including, but not limited to, any one or more of treating symptoms of myocardial infarction, infection or pain; or preventing myocardial infarction, infection or pain.

“Prevention” or “preventing” as used herein refers to any of: halting the effects of myocardial infarction, infection or pain; reducing the effects of myocardial infarction, infection or pain; reducing the incidence of myocardial infarction, infection or pain; reducing the development of myocardial infarction, infection or pain; delaying the onset of symptoms of myocardial infarction, infection or pain; increasing the time to onset of symptoms of myocardial infarction, infection or pain; and reducing the risk of development of myocardial infarction, infection or pain.

“Treatment” or “treating” as used herein refers to any of the alleviation, amelioration, elimination and/or stabilization of a symptom, as well as delay in progression of a symptom of a particular disorder. For example, “treatment” may include any one or more of the following: amelioration and/or elimination of one or more symptoms associated with myocardial infarction, infection or pain; reduction of one or more symptoms of myocardial infarction, infection or pain; stabilization of symptoms of myocardial infarction, infection or pain; and delay in progression of one or more symptoms of myocardial infarction, infection or pain.

“Infection” as used herein refers to the invasion of one or more microorganisms such as bacteria, viruses, fungi, yeast or parasites in the body of a patient in which they are not normally present.

“Antimicrobial” as used herein refers to natural or synthetic compositions capable of killing or inhibiting the growth of microorganisms including, but not limited to, bacteria, fungi, viruses, protozoa and parasites. Antimicrobials used herein include antibiotics, antivirals, antifungals, antiprotozoals, and antiparasitics.

“Anesthetics” as used herein refers to a natural or synthetic composition capable of producing a local, regional or general loss of sensation. Anesthetics are generally used to induce an insensitivity to pain. As used herein, the term “anesthetic” is used to refer to local anesthetics. Exemplary anesthetics that may be used herein include, but are not limited to, lidocaine, marcaine, bupivacaine, prilocaine, mepivacaine, etidocaine, ropivacaine, levobupivacaine.

“Growth factors” as used herein refers to a secreted biologically active molecule capable of affecting the growth of cells, promote or inhibit mitosis, or affect cellular differentiation. In some embodiments, growth factors include angiogenic an immunoregulatory proteins. Examples of growth factors that can be used as therapeutic agents in the present invention include, but are not limited to, vascular endothelial growth factor (VEGF); platelet-derived growth factor (PDGF); epidermal growth factor (EGF); granulocyte-macrophage colony-stimulating factor (GM-CSF); insulin-like growth factor 1 (IGF-1) and 2 (IGF-2); transforming growth factor (TGFα or TGFβ); fibroblast growth factor (FGF); cytokines such as interleukins (IL-1 through IL-18) and interferons (IFN-α, IFN-β, IFN-γ).

“Polymer” as used herein refers to a relatively high molecular weight organic compound, natural or synthetic, whose structure can be represented by a repeated small unit, the monomer. Synthetic polymers are typically formed by addition or condensation polymerization of monomers. Some combinations of polymers form the nanoparticles while other polymers or combinations thereof form the hydrogel. A polymer comprised of two or more different monomers is a copolymer.

In some embodiments, the polymers used to form the nanoparticles exist in a triblock copolymer having an A-B-A structure with polymer A being selected from the group including, but not limited to, polyethylene glycol (PEG), polycaprolactone (PCL), poly(glycolic acid) (PGA), poly(lactic acid) (PLA), poly(lactic acid-co-glycolic acid) (PLGA). Polymer B may be selected from the group including, but not limited to, poly(serinol hexamethylene urea) (PSHU). Exemplary triblock copolymers used to form the nanoparticles include, but are not limited to, PEG-PSHU-PEG, PCL-PSHU-PCL.

In some embodiments, the polymer used to form the hydrogel exists as a single polymer including, but not limited to, gelatin, collagen, chitosan, cellulose. In some embodiments, the diblock copolymer Poly(ethylene glycol diglycidyl ether) succinic acid (PEGSA) may be used. For use as a hydrogel, PEGSA needs to be crosslinked. Tyramine may be conjugated to PEGSA for the crosslinking reaction. Hydrogen peroxide or horseradish peroxidase may then be used to crosslink the ring groups on tyramine. In other embodiments, the polymers used to form the hydrogel exist as diblock copolymers having a C-D structure with polymer C being selected from the group including, but not limited to, poly(serinol hexamethylene urea) (PSHU), sulfonated poly(serinol hexamethylene urea) (SPSHU). Polymer D may be selected from the group including, but not limited to, poly(N-isopropylacrylamide) (PNIPAM). Exemplary diblock copolymers used to form the hydrogel include PSHU-PNIPAM, SPSHU-PNIPAM.

“Nanoparticle” as used herein refers to a particle or structure which is biocompatible with and sufficiently resistant to chemical and/or physical destruction by the environment of such use so that a sufficient number of the nanoparticles remain substantially intact after delivery to the site of application or treatment and whose size is in the nanometer range. For the purposes of the present invention, a nanoparticle typically ranges between about 1 nm to about 1000 nm, preferably between about 50 nm and about 500 nm, more preferably between about 50 nm and about 350 nm, more preferably between about 100 nm and about 250 nm. As used herein, the term “nanoparticle” includes, but is not limited to, micelles, polymeric nanoparticles, and lipid-based nanoparticles such as liposomes and niosomes. In some embodiments, a micelle is used as the preferred nanoparticle type.

“Crosslinking” as used herein refers to chemically joining two or more molecules by a covalent bond. Exemplary crosslinking agents include, but are not limited to, carbodiimides such as 1-Ethyl-3-(3-dimethylaminopropyl) carbodiimide (EDC) and dicyclohexylcarbodiimide (DCC); carbonyldiimidazole (CDI); genipin (GP) ß-glycerophosphate and glutaraldehyde; and the use of enzymes, including but not limited to transglutaminase, tyrosinases and horseradish peroxidases. Crosslinking agents can be introduced into the materials through physical crosslinking methods as well, including, but are not limited to, dehydrothermal and ultraviolet radiation treatment.

“Hydrogel” as used herein refers to a three-dimensional (3D) crosslinked network of biocompatible hydrophilic polymers having an elastic structure and a water content or at least 10%. The polymers may be naturally or synthetically derived. Hydrogels have the capacity to reversibly swell in water.

“Gelatin” as used herein refers to a water-soluble polymer obtained from acid, alkaline or enzymatic hydrolysis of collagen. Gelatin derived from acid treatment is referred to as type A while gelatin derived from alkaline treatment is referred to as type B. In some embodiments, the gelatin may be derived from porcine skin, bovine skin, bone, poultry, or fish. In other embodiments, the gelatin may be a recombinant gelatin.

“Catheter” as used herein refers to a hollow tube formed from a biocompatible material for insertion into body cavities to drain fluids, inject fluids or distend body passages. The hollow tube may be flexible or rigid depending on the use. “Drain” is used interchangeably herein with “catheter”. The term “catheter” includes, but is not limited to, pleural and mediastinal drains, as well as various drain types used in other anatomical spaces such as Blake drains, pigtail catheters, or Foley catheters. Exemplary pleural and mediastinal drains include, but are not limited to, chest tubes, pleural catheters, intercostal drains, thoracic catheters, and thoracostomy tubes.

The present disclosure provides drug delivery systems, components thereof, methods of use, methods of formation of the systems and components thereof as well as devices using such disclosed drug delivery systems.

The following non-limiting examples illustrate exemplary systems and components thereof in accordance with various embodiments of the disclosure. The examples are merely illustrative and are not intended to limit the disclosure in any way.

### Example 1—Formation of Therapeutic Agent-Loaded Micelles

Micelle Nanoparticle Polymer Synthesis: PEG-PSHU-PEG

NBOC serinol (0.2873 g, 1.5 mmol) and urea (0.09 g, 1.5 mmol) were weighed out and lyophilized at −45° C. for 48 h. The reactants were dissolved in 1.5 mL of anhydrous DMF in a 25 mL round-bottom flask at 90° C. under gentle stirring and a nitrogen atmosphere. HDI (0.482 mL, 3 mmol) was added dropwise, and the polymerization was carried out for 5 days. After the specified time, an excess of poly(ethylene glycol) (PEG 1000, 4 mmol) was dehydrated and added to the reaction. The PEGylation reaction was carried out for 24 h at 90° C. The resulting product, poly(ethylene glycol)-block-poly(serinol hexamethylene urea)-block-poly(ethylene glycol) (PEGPSHU-PEG), was purified by three precipitations in diethyl ether and then dried completely by extended rotary evaporation at 50° C. and 10 mbar vacuum. Subsequently, the polymer was dissolved in Milli-Q water and dialyzed (MWCO: 3.5 kDa) against 1 L of Milli-Q water for 72 h at room temperature. Then, the product was lyophilized at −45° C. for 48 h to yield a white flaky material.

Growth Factor-Loaded Micelle Fabrication (PEG-PSHU-PEG)

Growth factor-loaded micelles were fabricated by a traditional emulsification-sonication procedure. The PEG-PSHU-PEG polymer and growth factor were dissolved in 1 mL of DMSO at 3 wt % (polymer/DMSO). This solution was then added dropwise to a beaker containing 20 mL of ultrapure water partially submerged in an ultrasonic bath. The resulting emulsion was sonicated for 10 min. The removal of DMSO was carried out by centrifugation at 11,000 revolutions per minute (rpm) for 5 min, pouring off the supernatant, and then resuspending the micelles in ultrapure water. This DMSO extraction procedure was carried out three times. The resulting micelles were either used immediately or stored at −20° C. for later use.

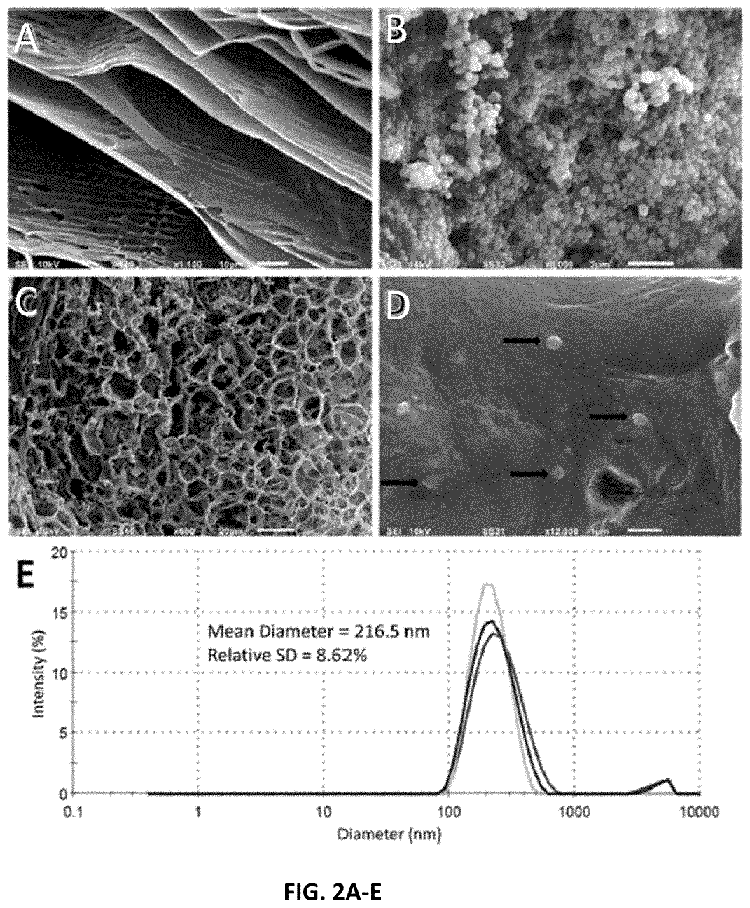

Novel micelle NPs were created from a PEG-PSHU-PEG polymer. The PSHU backbone provides the hydrophobic core of the micelles, while the terminal PEG chains provide the hydrophilic interactions on the exterior shell. PEG adds an additional benefit as this provides a “stealth” effect to the NPs for an extended half-life in circulation. The synthesis of PEG-PSHU-PEG from N—BOC serinol, urea, HDI, and PEG was confirmed using 1H NMR (500 MHz, DMSO-d6). The PSHU backbone structure was previously confirmed by 1H NMR,³⁴ and the successful conjugation of PEG to PSHU was confirmed by the chemical shift at δ 3.51 ppm (peak 1, —OCH2CH2O—), which identifies the protons on the PEG repeating unit of the polymer backbone.³⁵ Scanning electron microscopy (SEM) was used to characterize the morphologies of the micelles. The micelle NPs were uniformly spherical (FIG. 2B).

DLS is a technique that can be used to determine the size distribution profile of different particles, or polymers, in solution. The micelle NPs' size was determined using a Zetasizer Nano ZS. Three separate batches of micelles were made using the same protocol, and the particle size was measured (FIG. 2E). The DLS data are plotted using an intensity-weighted distribution. Micelles had a mean diameter of 216.5 nm with a relative standard deviation (RSD) of 8.62%. As shown with a low RSD value, the micelles are composed of a monodispersed population of particles and batch to batch variability appears to be low.

Lidocaine-Loaded Micelle Fabrication (PEG-PSHU-PEG)

Lidocaine-loaded micelles were fabricated by a traditional emulsification-sonication procedure. The PEG-PSHU-PEG polymer and lidocaine (10 mg polymer to 100 mg drug) were dissolved in 1 mL of DMSO at 3 wt % (polymer/DMSO). This solution was then added dropwise to a beaker containing 20 mL of ultrapure water partially submerged in an ultrasonic bath. The resulting emulsion was sonicated for 10 min. The removal of DMSO was carried out by centrifugation at 11,000 revolutions per minute (rpm) for 5 min, pouring off the supernatant, and then resuspending the micelles in ultrapure water. This DMSO extraction procedure was carried out three times. The resulting micelles were either used immediately or stored at −20° C. for later use.

While lidocaine was used in an embodiment, other anesthetic agents or antimicrobial agents are contemplated for use with the nanoparticles described herein.

Micelle size was determined using a Zetasizer Nano ZS.

Micelle Nanoparticle Polymer Synthesis: PCL-PSHU-PCL

N-Boc-serinol (1.149 g, 6 mmol) and urea (0.36 g, 6 mmol) were weighed out and lyophilized for 48 hours at −45° C. and 0.045 mbar. The reactants were dissolved in 10 mL of anhydrous DMF in a 25 mL round bottom flask at 90° C. under gentle stirring and a nitrogen atmosphere. Hexamethylene diisocyanate (HDI) (1.928 mL, 12 mmol) was added dropwise, and the polymerization was carried out for 3 days. After the specified time, 10% molar N-Boc-serinol (0.115 g, 0.6 mmol) was dissolved in anhydrous DMF and added to the reaction dropwise. The reaction was carried out for 24 h at 90° C. The resulting product, N-Boc-serinol-block-poly(serinol hexamethylene urea)-block-N-Boc-serinol (N-Boc-PSHU—N-Boc), was purified by three precipitations in diethyl ether and then dried completely by extended rotary evaporation at 50° C. and 10 mbar vacuum. Then the product was lyophilized for 48 h to yield a white flaky material and used for further synthesis. N-Boc-PSHU—N-Boc (0.5 g) was dissolved in 5 ml anhydrous DMF under a nitrogen atmosphere and stir. Polycaprolactone (PCL, 0.5 g) was added dropwise and a catalytic amount of stannous octoate (0.0025 g) was added to the flask, and the polymerization was carried out for 3 days at 70° C. The resulting product, polycaprolactone-block-poly(serinol hexamethylene urea)-block-polycaprolactone (PCL-PSHU-PCL), was purified by three precipitations in diethyl ether and then dried completely by extended rotary evaporation at 50° C. and 10 mbar vacuum. Then the product was lyophilized for 48 h to yield a white flaky material and used for further modification. PCL-PSHU-PCL can be deprotected by hydrogenation to provide free amine groups for additional chemical modification. However, micelles can be formed with or without this deprotection step. For the deprotection procedure, PCL-PSHU-PCL (0.1 g) was dissolved in DCM and TFA (9 ml each). Deprotection occurred by hydrogenation at room temperature for 15 min providing free amine groups. Deprotected PSHU-PCL (PCL-dPSHU-PCL) was recovered by precipitation in diethyl ether and rotoevaporation. Further purification of dPSHU involved dissolving in TFE, precipitation in diethyl ether, and rotoevaporation. The product was lyophilized for 48 h.

Lidocaine-loaded micelles were fabricated by a traditional emulsification-sonication procedure. The PCL-PSHU-PCL polymer and lidocaine (10 mg polymer to 100 mg drug) were dissolved in 1 mL DMSO at 1 wt % (polymer/DMSO). This solution was then added dropwise to a beaker containing 20 mL of purified water (milliQ) partially submerged in an ultrasonic bath. The resulting emulsion was sonicated for 10 min. Removal of DMSO was carried out by centrifugation at 11,000 revolutions per minute (rpm) for 5 min, pouring off the supernatant and then re-suspending the micelles in purified water. This DMSO extraction procedure was carried out 3 times. The resulting micelles were used immediately.

### Example 2—Formation of Hydrogel

Synthesis of Crosslinked Gelatin Hydrogel

An aqueous solution containing gelatin was prepared in PBS (5% w/v) and allowed to dissolve overnight in a 37° C. water bath. In embodiments in which lidocaine is loaded directly into the hydrogel, lidocaine-HCl was dissolved in the gelatin solution (200 mg lidocaine/1 ml gelatin solution). Glutaraldehyde was then added to the gelatin-lidocaine mixture (1% w/v) to crosslink the gelatin into a hydrogel. While 1% w/v of crosslinker was used, a range of between about 0.5% to about 4% w/v is contemplated.

Synthesis of Sulfonated Reversible Thermal Gel: SRTG

NBOC serinol was synthesized as described previously.⁸ Briefly, serinol and di-tert-butyl dicarbonate were dissolved in ethanol at 4° C. The solution was heated at 37° C. for 1 h, roto-evaporated, and dissolved in an equal volume mixture of ethyl acetate and hexane at 60° C. Additional hexane was added to form crystalline structures, and the precipitate was filtered to remove solvent, yielding N—BOC serinol as a crystalline white product.

PNIPAM was synthesized as described previously.³⁰ In short, NIPAM and 4,4-azobis(4-cyanovaleric acid) were dissolved in methanol and heated at 68° C. for 3 h. PNIPAM was recovered by precipitation in ultrapure water at 60° C., purified via dialysis (MWCO 12-14 kDa), and lyophilized, yielding a white product.

SRTG (SPSHU-PNIPAM) was synthesized similarly as described previously.³¹ N—BOC serinol, urea, and HDI were dissolved in DMF and heated at 90° C. for 7 days. PSHU was recovered by precipitation in diethyl ether and rotoevaporation, yielding the polyurea as a white powder. PSHU was dissolved in DCM and TFA. Deprotection occurred by hydrogenation at room temperature for 45 min providing, free amine groups. Deprotected PSHU (dPSHU) was recovered by precipitation in diethyl ether and rotoevaporation. Further purification of dPSHU involved dissolving in 2,2,2-trifluoroethanol, precipitation in diethyl ether, and rotoevaporation. Next, an equivalent mass of PNIPAM was conjugated to dPSHU. PNIPAM, EDC, and NHS were dissolved in DMF and activated for 24 h. Separately, dPSHU was dissolved in DMF, added to the activated PNIPAM solution, and reacted for 24 h. PSHU-PNIPAM was recovered by precipitation in diethyl ether, rotoevaporation, purified via dialysis (MWCO 12-14 kDa), and lyophilized. Afterward, the remaining free amine groups were sulfonated. 1,3-Propane sultone, potassium tert-butoxide, and PSHU-PNIPAM were dissolved in DMF and reacted at 60° C. for 3 days. SRTG was recovered by precipitation in diethyl ether, rotoevaporation, purified via dialysis (MWCO 12-14 kDa), and lyophilized, yielding a light-yellow product. Polymer characterization was performed as described previously to confirm material produced.^{5, 9, 32}

Scanning Electron Microscopy (SEM)

SRTG samples (5%, phosphate-buffered saline (PBS)) were gelled at 37° C. for 15 min. The hydrogels were snap-frozen in liquid nitrogen. The frozen samples were quickly broken to expose the structure within the scaffold and lyophilized. Samples were sputtercoated with gold and palladium and examined by SEM. Dynamic Light Scattering (DLS).

Scanning electron microscopy (SEM) was used to characterize the morphologies of the SRTG, the growth factor-loaded micelles, and the combined delivery system, which was fabricated by simply mixing the growth-factor loaded micelles into SRTG polymer solution before gelation. The SRTG alone is composed of polymer sheets (FIG. 2A), while the micelle NPs were uniformly spherical (FIG. 2B). FIG. 2C shows the morphology of the SRTG encapsulating the micelle NPs. The morphology of the polymeric gel embedded with micelles is substantially altered from the original polymer sheets, as the cross-sectional image shows the gel in a highly porous configuration. FIG. 2D is an enlargement of the porous area of the same polymeric gel showing that the micelles are embedded within the gel and their uniform spherical shape has been maintained. The black arrows indicate the micelles in the gel.

### Example 3—Delivery of Growth Factor-Loaded Micelles in Sulfonated Reverse Thermal Gel

In this embodiment, the inventors demonstrate the efficacy of the SRTG micelle polymer delivery system for the controlled release of proteins to promote therapeutic angiogenesis and reduce inflammation. PEG-PSHU-PEG polymeric micelles were synthesized and characterized for chemical structure and morphology. The PDGF-loaded micelles were encapsulated into the SRTG and examined for sequential and sustained release of the growth factor. Additionally, VEGF and IL-10 were encapsulated within the thermal gel system and their spatiotemporal release was evaluated. An in vitro release test revealed that the delivery system is sequentially releasing all three factors and at a sustained rate for 28 days. An in vivo subcutaneous injection mouse model showed that injection groups with both VEGF and PDGF-loaded micelles exhibited a significant increase in mature blood vessel formation compared to the dual growth factor group without micelles. Therefore, this validates the importance of sequentially delivering growth factors involved in distinct stages of angiogenesis. When IL-10 was loaded into the system with the growth factors, the inflammatory response to a foreign body revealed a significant decrease in macrophage presence compared to the other polymer injection groups. These results demonstrate that the SRTG micelle system can effectively deliver proteins in a controlled manner for therapeutic angiogenesis and could potentially reduce harmful pathological remodeling post-MI.

Materials and Methods

Materials

N-Isopropylacrylamide (NIPAm) was purchased from Tokyo Chemical Industry (Chuo-ku, Tokyo, Japan). Anhydrous N,Ndimethylformamide (DMF) was purchased from EMD Millipore (Billerica, Mass.). 4,4′-Azobis(4-cyanovaleric acid) (ACA), N-hydroxysuccinimide (NHS), sodium bicarbonate, 1-bromohexane, TWEEN 20, Triton™ X-100, 7-globulins from bovine blood, urea, hexamethylene diisocyante (HDI), acetic acid, sodium acetate, 1,3-propane sultone (PS), trifluoroacetic acid (TFA), triethylamine, dimethyl sulfoxide (DMSO), 2,2′-azino-bis(3-ethylbenzothiazoline-6-sulfonic acid) (ABTS) liquid substrate system, dichloromethane (DCM), and bovine serum albumin (BSA) were purchased from Sigma-Aldrich (St. Louis, Mo.). Di-tert-butyl dicarbonate, ethyl acetate, Nhydroxysuccinimide (NHS), N-(3-dimethylamino-propyl)-N′-ethylcarbodiimide hydrochloride (EDC), 2-amino-1,2-propanediol, 98% (Serinol), anhydrous methanol, dimethyl sulfoxide-d6 (DMSO-d6), poly(ethylene glycol) 1000, ethanol, hexane, and anhydrous diethyl ether were purchased from Fisher Scientific (Pittsburgh, Pa.). Goat anti-Rabbit IgG (H+L) Secondary Antibody Alexa Fluor 594, Goat anti-Rat IgG (H+L) Secondary Antibody Alexa Fluor 488, Rabbit anti-Goat IgG (H+L) Secondary Antibody Alexa Fluor 594, CD31 Antibody (Rat IgG2a), and smooth muscle actin antibody (α-SMA, rabbit IgG) were purchased from Thermo Fisher Scientific (Waltham, Mass.). Anti-Von Willebrand factor antibody (VWF, sheep IgG) and anti-CD68 antibody (rabbit IgG) were purchased from Abeam (Cambridge, U.K.). Human PDGF-BB Standard ABTS ELISA development kit, Murine IL-10 Standard ABTS ELISA development kit, Murine VEGF Standard ABTS ELISA development kit, Recombinant Human and Murine PDGF-BB, Recombinant Murine IL-10, and Recombinant Murine VEGF165 were purchased from Peprotech (Rocky Hill, N.J.). 10% formalin was purchased from JT Baker (Phillipsburg, N.J.). Sucrose (RNASE and DNASE free) was purchased from VWR Life Science (Radnor, Pa.). An optimal cutting temperature (OCT) compound was purchased from Sakura (Torrance, Calif.). Alexa Fluor 594 (goat anti-rabbit IgG) was purchased from Life Technologies (Carlsbad, Calif.). DAPI Flouromount-G was purchased from Electron Microscope Sciences (Hartfield, Pa.). Spectra/Por dialysis membranes (molecular weight cut-off (MWCO): 3500-5000 and 12 000-14 000 Da) were purchased from Spectrum Laboratories (Rancho Dominguez, Calif.).

Equipment

Proton nuclear magnetic resonance (1H NMR) was performed on a Varian Inova 500 NMR spectrometer, and samples were run in DMSO-d6 at room temperature. Polymer morphology was imaged using a JEOL (Peabody, Mass.) JSAM-6010LA analytical scanning electron microscope. Nanoparticle size was measured using a Zetasizer Nano ZS (Malvern Instruments Ltd, Worcestershire, U.K.). ELISA color development was monitored with a BioTek Synergy 2 Multi-Mode Reader at 405 nm with wavelength correction set at 650 nm. Confocal images were taken using the Zeiss LSM 780.

Synthesis of Sulfonated Reversible Thermal Gel: SRTG

NBOC serinol was synthesized as described previously.⁸ Briefly, serinol and di-tert-butyl dicarbonate were dissolved in ethanol at 4° C. The solution was heated at 37° C. for 1 h, rotoevaporated, and dissolved in an equal volume mixture of ethyl acetate and hexane at 60° C. Additional hexane was added to form crystalline structures, and the precipitate was filtered to remove solvent, yielding N—BOC serinol as a crystalline white product.

PNIPAM was synthesized as described previously.³⁰ In short, NIPAM and 4,4-azobis(4-cyanovaleric acid) were dissolved in methanol and heated at 68° C. for 3 h. PNIPAM was recovered by precipitation in ultrapure water at 60° C., purified via dialysis (MWCO 12-14 kDa), and lyophilized, yielding a white product.

SRTG (SPSHU-PNIPAM) was synthesized similarly as described previously.³¹ N—BOC serinol, urea, and HDI were dissolved in DMF and heated at 90° C. for 7 days. PSHU was recovered by precipitation in diethyl ether and rotoevaporation, yielding the polyurea as a white powder. PSHU was dissolved in DCM and TFA. Deprotection occurred by hydrogenation at room temperature for 45 min providing, free amine groups. Deprotected PSHU (dPSHU) was recovered by precipitation in diethyl ether and rotoevaporation. Further purification of dPSHU involved dissolving in 2,2,2-trifluoroethanol, precipitation in diethyl ether, and rotoevaporation. Next, an equivalent mass of PNIPAM was conjugated to dPSHU. PNIPAM, EDC, and NHS were dissolved in DMF and activated for 24 h. Separately, dPSHU was dissolved in DMF, added to the activated PNIPAM solution, and reacted for 24 h. PSHU-PNIPAM was recovered by precipitation in diethyl ether, rotoevaporation, purified via dialysis (MWCO 12-14 kDa), and lyophilized. Afterward, the remaining free amine groups were sulfonated. 1,3-Propane sultone, potassium tert-butoxide, and PSHU-PNIPAM were dissolved in DMF and reacted at 60° C. for 3 days. SRTG was recovered by precipitation in diethyl ether, rotoevaporation, purified via dialysis (MWCO 12-14 kDa), and lyophilized, yielding a light-yellow product. Polymer characterization was performed as described previously to confirm material produced.^{5, 9, 32}

Micelle Nanoparticle Polymer Synthesis: PEG-PSHU-PEG

NBOC serinol (0.2873 g, 1.5 mmol) and urea (0.09 g, 1.5 mmol) were weighed out and lyophilized at −45° C. for 48 h. The reactants were dissolved in 1.5 mL of anhydrous DMF in a 25 mL round-bottom flask at 90° C. under gentle stirring and a nitrogen atmosphere. HDI (0.482 mL, 3 mmol) was added dropwise, and the polymerization was carried out for 5 days. After the specified time, an excess of poly(ethylene glycol) (PEG 1000, 4 mmol) was dehydrated and added to the reaction. The PEGylation reaction was carried out for 24 h at 90° C. The resulting product, poly(ethylene glycol)-blockpoly(serinol hexamethylene urea)-block-poly(ethylene glycol) (PEGPSHU-PEG), was purified by three precipitations in diethyl ether and then dried completely by extended rotary evaporation at 50° C. and 10 mbar vacuum. Subsequently, the polymer was dissolved in Milli-Q water and dialyzed (MWCO: 3.5 kDa) against 1 L of Milli-Q water for 72 h at room temperature. Then, the product was lyophilized at −45° C. for 48 h to yield a white flaky material.

Micelle Fabrication

Growth factor-loaded micelles were fabricated by a traditional emulsification-sonication procedure. The PEG-PSHU-PEG polymer and growth factor were dissolved in 1 mL of DMSO at 3 wt % (polymer/DMSO). This solution was then added dropwise to a beaker containing 20 mL of ultrapure water partially submerged in an ultrasonic bath. The resulting emulsion was sonicated for 10 min. The removal of DMSO was carried out by centrifugation at 11,000 revolutions per minute (rpm) for 5 min, pouring off the supernatant, and then resuspending the micelles in ultrapure water. This DMSO extraction procedure was carried out three times. The resulting micelles were either used immediately or stored at −20° C. for later use.

Scanning Electron Microscopy (SEM)

SRTG samples (5%, phosphate-buffered saline (PBS)), with and without micelles, were gelled at 37° C. for 15 min. The hydrogels were snap-frozen in liquid nitrogen. The frozen samples were quickly broken to expose the structure within the scaffold and lyophilized. Samples were sputtercoated with gold and palladium and examined by SEM. Dynamic Light Scattering (DLS). The micelle's size was determined using a Zetasizer Nano ZS. Three separate batches of micelles were made using the same protocol, and the particle size was measured.

Proton Nuclear Magnetic Resonance (1H NMR)

Proton nuclear magnetic resonance (1H NMR) was completed with a Varian Inova 500 NMR spectrometer. 1H NMR was used to confirm the structure of PEG-PSHU-PEG. The PEG-PSHU-PEG samples to be analyzed were dissolved in 600 μL of DMSO-d6. Protein Release Test. SRTG 5 wt % polymeric solutions (SRTG+VEGF+NPs+PDGF+IL-10) were created using PBS with 0.2% BSA with samples run in triplicates. Solutions were mixed and left to dissolve overnight at 4° C. 500 ng of each of IL-10, the VEGF, and the PDGF was added to the gels at different times. The micelle NPs loaded with the PDGF were added 30 min before gelling, and the VEGF and IL-10 were added 10 min before gelation. 50 μL gels were formed in 2 mL vials and placed in a 37° C. incubator for 5 min to promote gelation and encapsulation of proteins and micelles. Then, 1 mL of warm (37° C.) release solution (lx PBS with 0.2% BSA) was added to the vials. After 5 min, 1 mL of release solution was removed from each sample for the day 0 time point, and 1 mL of fresh warm release solution was added. Samples were then taken every 24 h and immediately placed in a −80° C. freezer for further analysis. Enzyme-linked immunosorbent assay (ELISA) was used to quantify the concentration of proteins in the release solution samples following the manufacturer's instructions (Peprotech, Rocky Hill, N.J.). ELISA color development was monitored with a BioTek Synergy 2 Multi-Mode Reader at 405 nm with wavelength correction set at 650 nm. The results of three replicates were averaged. The hydrogels were initially allowed to stabilize for 5 min at which a sample was taken to determine the loading efficiency of the proteins in the hydrogels (day 0).

Subcutaneous Injection in the Mouse Model

Animal procedures were approved by the Institutional Animal Care and Use Committee (IACUC). C57BL/6J mice (The Jackson Laboratory) weighing 24-28 g were maintained on a light/dark (14 h light, 10 h dark) cycle with access to food and water ad libitum. The mice were anesthetized using continuous isoflurane and oxygen inhalation. Initial induction was at 5% isoflurane in oxygen and then maintained at 2% isoflurane in oxygen.

A total of 36 adult male C57BL/6 mice were used for the study, 3 mice per each injection group (saline, SRTG, SRTG+VEGF, SRTG+VEGF+PDGF, SRTG+VEGF+NPs+PDGF, or SRTG+VEGF+NPs+PDGF+IL-10) for 2 time points (7, 21 days). The mice were allowed 7 days to acclimate prior to injections. Proteins were loaded as described in the protein release test section. Saline (60 μL of saline), SRTG (1%, 60 μL of saline), SRTG+VEGF (250 ng, 60 μL of saline), SRTG+VEGF+PDGF (1%, 250 ng of VEGF and PDGF each, 60 μL of saline), SRTG+VEGF+NPs+PDGF (1%, 250 ng of VEGF and PDGF each, 3 mg of NPs, 60 μL of saline), or SRTG+VEGF+NPs+PDGF+IL-10 (1%, 250 ng of VEGF, PDGF, and IL-10 each, 3 mg of NPs, 60 μL of saline) was injected subcutaneously in the lower back of the mice through a 27-gauge needle.

Subcutaneous Tissue Harvest

The mice were euthanized by carbon dioxide and cervical dislocation 7 or 21 days after injection. After removing the hair around the injection site with depilatory cream, the subcutaneous tissue (2 cm×2 cm) was harvested at the site of injection. The site was clearly visible as a result of shaving the backs of the mice at the time of the initial injection. The subcutaneous tissue was fixed in formalin (10%, PBS) overnight, cryoprotected with sucrose (30%, PBS) for 1 day, embedded in optimal cutting temperature (OCT) compound, and frozen at −80° C. The subcutaneous tissue was sectioned transversely with a thickness of 5 μm.

Immunohistochemistry

Sections were fixed in formalin (10%, PBS) for 10 min and washed three times with wash buffer (0.1% Tween 20, PBS) for 5 min each. Permeabilization buffer (0.5% Triton X-100, PBS) was used for 10 min, and the sections were washed three times with a wash buffer for 5 min each. Blocking buffer (0.25% Triton X-100, 2% bovine serum albumin (BSA), 4% bovine 7 globulins, and PBS) was used for 60 min on the sections at room temperature. All antibodies were diluted in dilution buffer (0.25% Triton X-100, 2% BSA, 4% bovine 7 globulins, and PBS). The sections were stained with primary antibodies CD31 (1:50), VWF (1:50), SMA (1:250), and/or CD68 (1:500) overnight at 4° C. and washed three times in wash buffer for 5 min each. The sections were stained with secondary antibodies Alexa Fluor 488 (1:500) for CD31 and/or the associated Alexa Fluor 594 (1:500) for VWF, SMA, and CD68 at room temperature. The sections were washed three times in wash buffer for 5 min each and washed three times in ultrapure water for 5 min each. DAPI Fluoromount-G was used to stain nuclei and mount the sections. Cell and vessel counts from confocal images of the immunohistochemically stained subcutaneous tissue were obtained at three random visual fields with z-stack projections (4 m thickness, 1 m steps) and averaged. Visual fields of saline group injections were taken around the center of the tissue section, while visual fields with hydrogel injections were taken just outside the area of hydrogel integration with the subcutaneous tissue. Five low-magnification (200×) fields containing the highest number of CD31-, α-SMA-, VWF-, or CD68-positive cells were selected for each group following previously published criteria.³³ The number of CD31-, α-SMA-, VWF-, or CD68-positive cells in the field was counted and confirmed by DAPI-positive nuclei.

Statistical Analysis

Two-tailed t-test assuming unequal variance was used to determine significant differences between two groups. Analysis of variance (ANOVA) was used to determine significant differences between three or more groups followed by Tukey's test to determine significant differences between two groups as appropriate. Statistical significance was considered when p<0.05.

Results

Synthesis of PEG-PSHU-PEG and Micelle Fabrication and Characterization

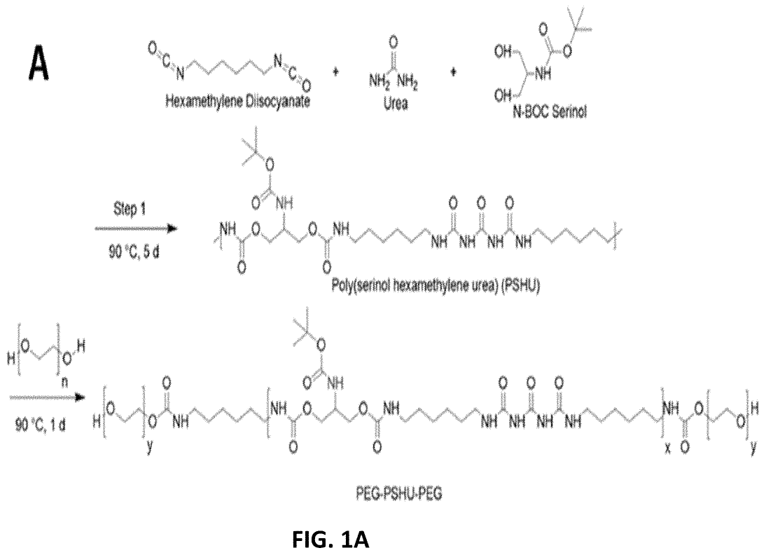

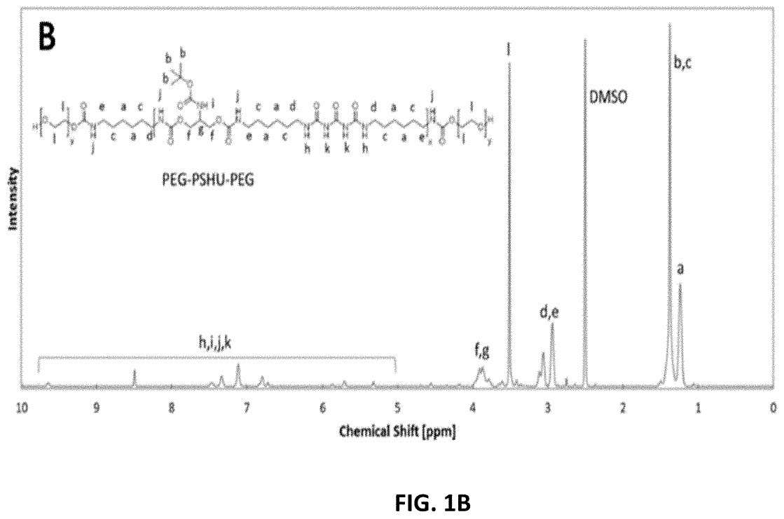

Novel micelle NPs were created from a PEG-PSHU-PEG polymer, which is based on the same PSHU backbone as SRTG, making the polymer biocompatible and biodegradable. As this polymer is used to fabricate micelles, it must have differing sections of hydrophobic and hydrophilic chains to form these particles. The PSHU backbone provides the hydrophobic core of the micelles, while the terminal PEG chains provide the hydrophilic interactions on the exterior shell, which can ionically bind to growth factors (FIG. 1A). PEG adds an additional benefit as this provides a “stealth” effect to the NPs for an extended half-life in circulation. When the micelles are injected together with SRTG, the micelles can provide long-term and sequential release of angiogenic growth factors, such as PDGF, while the SRTG provides a scaffold for their retention at the injection site. The synthesis of PEG-PSHU-PEG from N—BOC serinol, urea, HDI, and PEG was confirmed using 1H NMR (500 MHz, DMSO-d6). The 1H NMR spectrum is shown in FIG. 1B. The PSHU backbone structure was previously confirmed by 1H NMR,³⁴ and the successful conjugation of PEG to PSHU was confirmed by the chemical shift at δ 3.51 ppm (peak 1, —OCH2CH2O—), which identifies the protons on the PEG repeating unit of the polymer backbone.³⁵ Scanning electron microscopy (SEM) was used to characterize the morphologies of the SRTG, micelles, and the combined delivery system. The SRTG alone is composed of polymer sheets (FIG. 2A), while the micelle NPs were uniformly spherical (FIG. 2B). FIG. 2C shows the morphology of the SRTG encapsulating the micelle NPs. The morphology of the polymeric gel embedded with micelles is substantially altered from the original polymer sheets, as the cross-sectional image shows the gel in a highly porous configuration. FIG. 2D is an enlargement of the porous area of the same polymeric gel showing that the micelles are embedded within the gel and their uniform spherical shape has been maintained. The black arrows indicate the micelles in the gel.

DLS is a technique that can be used to determine the size distribution profile of different particles, or polymers, in solution. The micelle NPs' size was determined using a Zetasizer Nano ZS. Three separate batches of micelles were made using the same protocol, and the particle size was measured (FIG. 2E). The DLS data are plotted using an intensity-weighted distribution. Micelles had a mean diameter of 216.5 nm with a relative standard deviation (RSD) of 8.62%. As shown with a low RSD value, the micelles are composed of a monodispersed population of particles and batch to batch variability appears to be low.

SRTG Micelle Polymer System Delivers Proteins in a Sequential and Sustained Manner

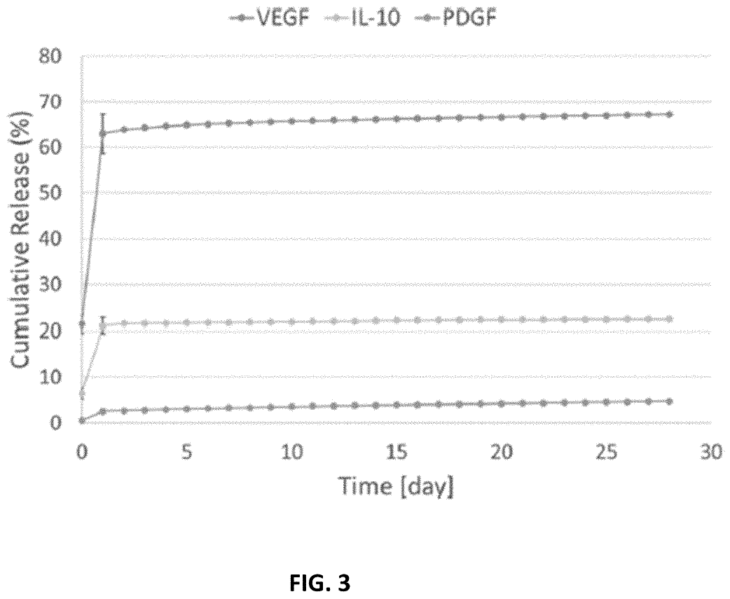

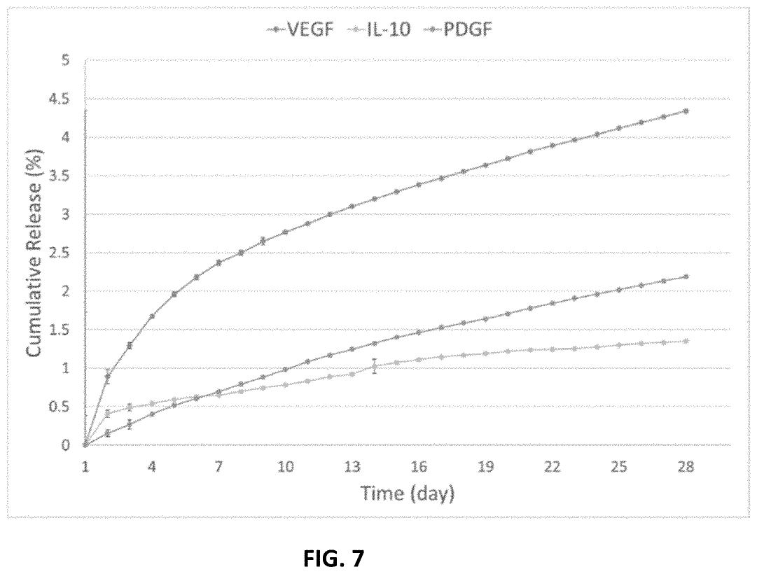

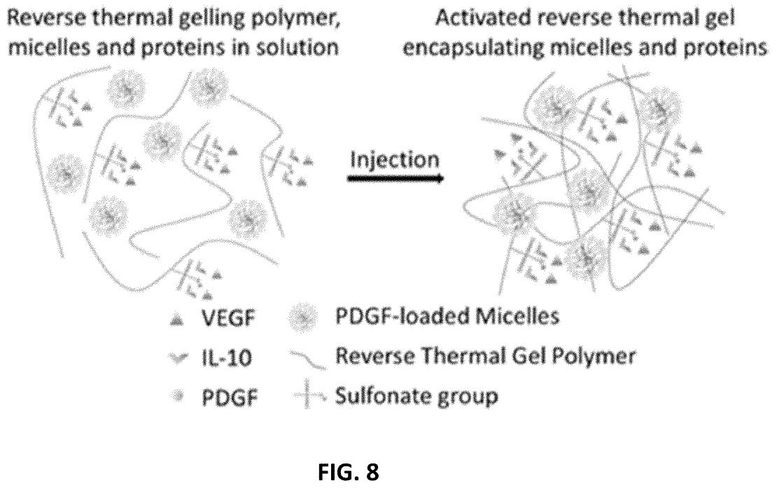

An in vitro release test study was performed to examine the sequential and sustained release rates of the SRTG loaded with the three proteins and micelles. According to the literature, IL-10 has a high heparin binding affinity and a similar affinity compared to FGF-2 (IL-10: Kd=54 nM;²⁶ FGF-2: Kd=74 nM³⁶). Additionally, it has been shown that FGF-2 has a much stronger binding affinity for heparin than VEGF (FGF-2: KA=1.68×107; VEGF: KA=8.14×105),³⁷ which would indicate that IL-10 should have a stronger binding affinity for the sulfonate groups on SRTG. Based on these binding affinities for heparin, it was hypothesized that VEGF would be released from the SRTG system first, followed by IL-10. As these two proteins were only mixed into the polymer solution, they should bind to the sulfonate groups according to their electrostatic binding affinities. PDGF should be released from the scaffold last as this growth factor is loaded into the micelles and embedded in the thermal gel. The release profile for the SRTG shows that the sulfonate groups and micelles are providing sustained and sequential release of the proteins (FIG. 3). The loading efficiencies were 79% for VEGF, 93% for IL-10, and 99% for PDGF (FIG. 3). By day 1, approximately 63% of the loaded VEGF was released, while the overall sustained release rate after day 0 was 0.77 ng per day through 28 days (FIG. 3, orange). For IL-10, there was a reduced burst release compared to VEGF, likely from the stronger binding affinity to the sulfonate groups, with approximately 21% released by day 1 and a sustained release rate of 0.24 ng per day from day 1 to 28 days (FIG. 3, yellow). PDGF demonstrated a low burst release at day 1 as approximately 3% of the growth factor released from the micelles (FIG. 3, green) and had a sustained release rate of 0.39 ng per day after day 0. A modified version of this release profile has been included (FIG. 7) to better illustrate the data since it can be difficult to discern the release rates from the full graph. This demonstrates that the sulfonate groups on the SRTG are mimicking the growth factor binding function of heparin by electrostatically binding to IL-10 to reduce its release rate compared to VEGF. Furthermore, the different heparin-binding affinities between VEGF and IL-10, in combination with the PDGF-loaded micelles, appear to be achieving the sequential release of all three proteins. This sequential release is a critical component of the delivery system for it to be applied toward a future treatment for MI.

Sequential Delivery of VEGF and PDGF Increased Functional Vascular Endothelial Cell Recruitment and Endothelial Cell Differentiation into Vascular Cells

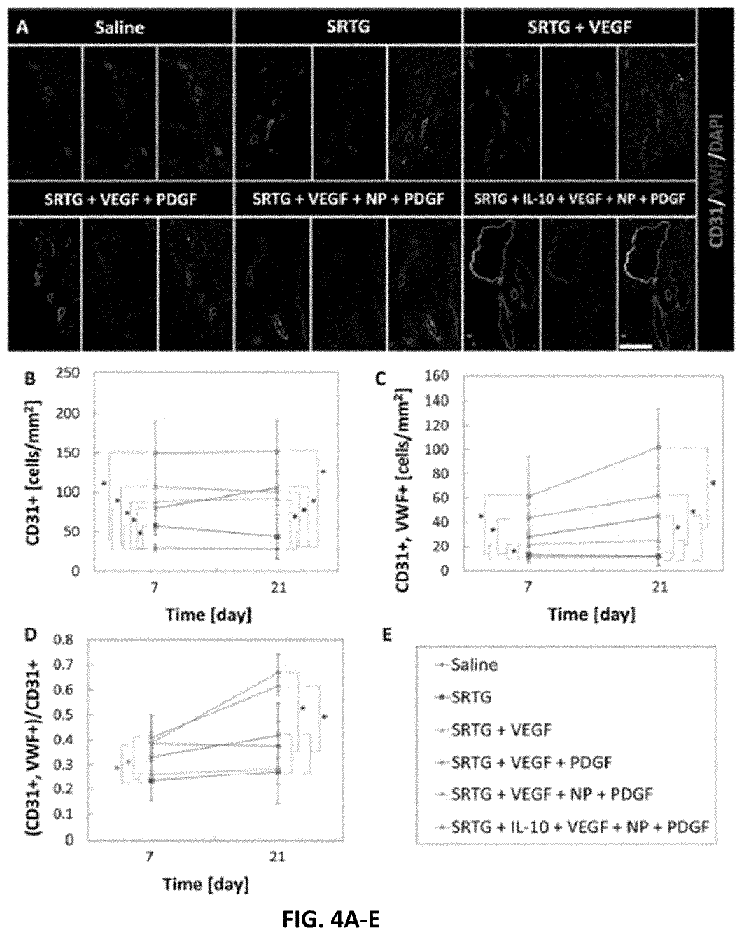

To assess the angiogenesis response efficacy in vivo, the SRTG polymeric delivery system was subcutaneously injected in the middle back of mice. The process of angiogenesis encompasses protein binding to endothelial cell receptors, basement membrane degradation by matrix metalloproteinases, endothelial cell proliferation and migration, subsequent vessel formation and remodeling, and finally vessel stabilization by pericytes.³⁸ Immunohistochemistry was performed to directly identify functional blood vessels. This staining technique allows for the quantification of the different cell types involved in the process of blood vessel formation. Endothelial cells were stained with a CD31 antibody, as this protein makes up a substantial portion of endothelial cell intercellular junctions. VWF, a factor involved in hemostasis, was used to identify functional vascular endothelial cells when endothelial cells were stained with both VWF and CD31 (FIG. 4).

Staining for CD31 was first examined to compare the proliferation and recruitment of endothelial cells associated with blood vessel lumen formation, as this is the initial step in the angiogenesis process. After 7 days, all five SRTG groups showed significantly more endothelial cell recruitment (CD31+ cells) than saline (FIG. 4B). After 21 days, all four SRTG groups loaded with proteins showed significantly more endothelial cells than saline (FIG. 4B). The SRTG group, without any factors, showed significantly more endothelial cells recruited at 7 days compared to saline, which can be attributed to an immune response induced by the polymer injection. It has been shown that tumor necrosis factor-α (TNF-α) is secreted by activated macrophages during inflammation and immune response.³⁹ TNF-α then acts on endothelial cells by inducing cell differentiation and by stimulating the production of angiogenic factors from other cells.⁴⁰ This inflammatory response pathway for promoting vascularization due to a foreign body was confirmed by the previous studies.⁵ Additionally, the SRTG+VEGF+NPs+PDGF+IL-10 group displayed significantly more endothelial cells than saline, SRTG, and SRTG+VEGF+PDGF after 7 days, and it showed significantly more endothelial cells than saline and SRTG after 21 days (FIG. 4B). This demonstrates that the sustained release of proteins from the polymer delivery system stimulates the proliferation of endothelial cells. Co-staining for CD31 and VWF was examined to quantify the amount of functional vascular endothelial cells within blood vessels, as this indicates endothelial cell differentiation into functional vascular cells. After 7 and 21 days, the SRTG+VEGF+NPs+PDGF+IL-10 and SRTG+VEGF+NPs+PDGF groups showed significantly more functional endothelial cells (CD31+ and VWF+ cells) than saline, SRTG, and SRTG+VEGF (FIG. 4C). The SRTG+VEGF group did not show a statistically significant difference in functional endothelial cells compared to the SRTG and saline groups, which shows that the delivery of PDGF is important to increase the proliferation of vascular endothelial cells (FIG. 4C).

Furthermore, the ratio of functional vascular endothelial cells to endothelial cells was quantified to determine the number of endothelial cells maturing into vascular cells. Both sequential delivery groups, with the PDGF-loaded micelle NPs, demonstrated a significant increase in functional vascular endothelial cell maturation after 21 days, while the other groups did not (FIG. 4D). This highlights the importance of sequentially delivering different angiogenic factors that are involved in distinct stages of the vessel formation process to promote more mature and functional vessels.

Sequential Delivery of VEGF and PDGF Improved Mature Blood Vessel Formation by Increasing Vascular Smooth Muscle Cell Recruitment

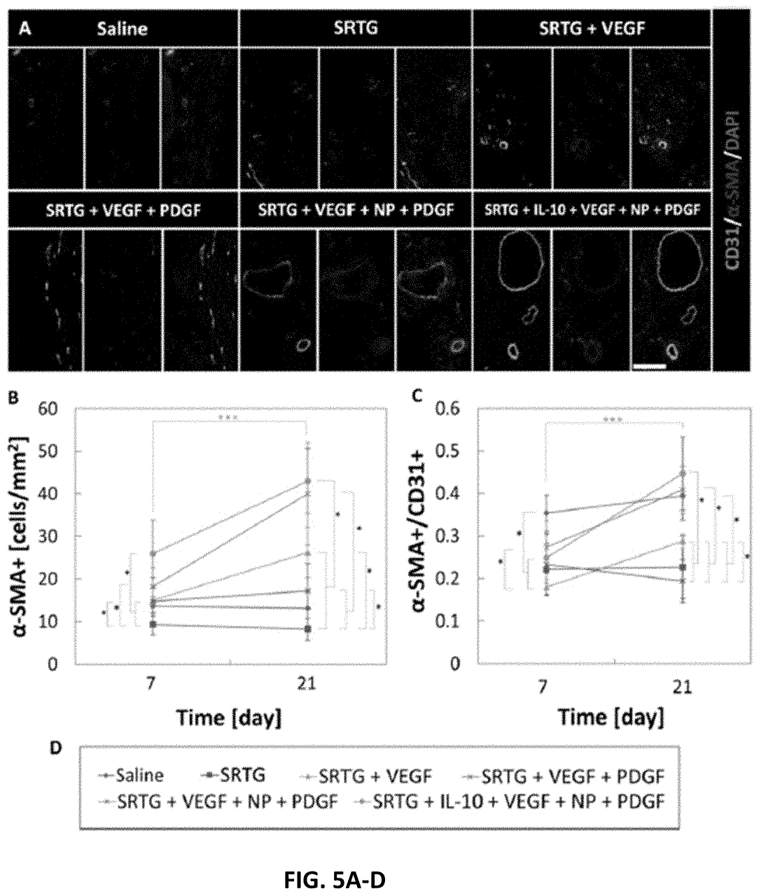

The recruitment of pericytes (mural cells or vascular smooth muscle cells) was used to identify stable and mature vasculature. Newly formed vessels can become hemorrhagic and may regress over time if they are not stabilized with recruited pericytes.^{14, 20, 41} Immunohistochemistry was implemented to observe the recruitment of pericytes to endothelial cell vessel lumens. Pericytes were stained with α-smooth muscle actin (α-SMA), which is a major component of microfilament bundles responsible for contractile functions. The association of α-SMA+ cells around CD31+ cells was used to identify mature and stable blood vessels at 7 and 21 days after injection (FIG. 5). More mature vessels should show an organized circle of endothelial cells surrounded by vascular smooth muscle cells.

After 7 days, SRTG+VEGF+NPs+PDGF+IL-10 recruited significantly more pericyte cells than saline, SRTG, and SRTG+VEGF groups (FIG. 5B). After 21 days, the two sequential delivery groups, SRTG+VEGF+NPs+PDGF+IL-10 and SRTG+VEGF+NPs+PDGF, displayed significantly more pericyte recruitment compared with saline, SRTG, and SRTG+VEGF+PDGF groups (FIG. 5B). Additionally, the SRTG+VEGF, SRTG+VEGF+NPs+PDGF, and SRTG+VEGF+NPs+PDGF+IL-10 groups all recruited significantly more pericytes over time when comparing the two time points (FIG. 5B). This demonstrates that the growth factors are inducing more mature neovasculature from the early to late stages of the angiogenesis process.

The SRTG+VEGF+PDGF group did not show any increase in pericyte recruitment between 7 and 21 days, unlike the other delivery systems loaded with growth factors (FIG. 5B). This group does not have PDGF encapsulated within NPs, which indicates that VEGF and PDGF are most likely burst releasing simultaneously. As stated earlier, it has been shown that early-stage angiogenic factors can have inhibitory effects on late-stage growth factors and vice versa, when presented concurrently.^{15-17, 20} These results for the SRTG+VEGF+PDGF group further confirm the importance of sequentially delivering angiogenic growth factors to develop stable and mature blood vessels. Moreover, both sequential delivery groups showed a significant increase in the ratio of vascular smooth muscle cells to endothelial cells compared to that of the other three polymer injection groups after 21 days (FIG. 5C). The ratio approached the physiological ratio shown in the saline control, as would be expected. The lack of vessel stabilization and maturation observed with the other growth factor delivery groups shows the limitations of VEGF delivery without the sequential release of a growth factor to recruit pericytes.

Delivery of IL-10 from SRTG Decreased Macrophage Infiltration

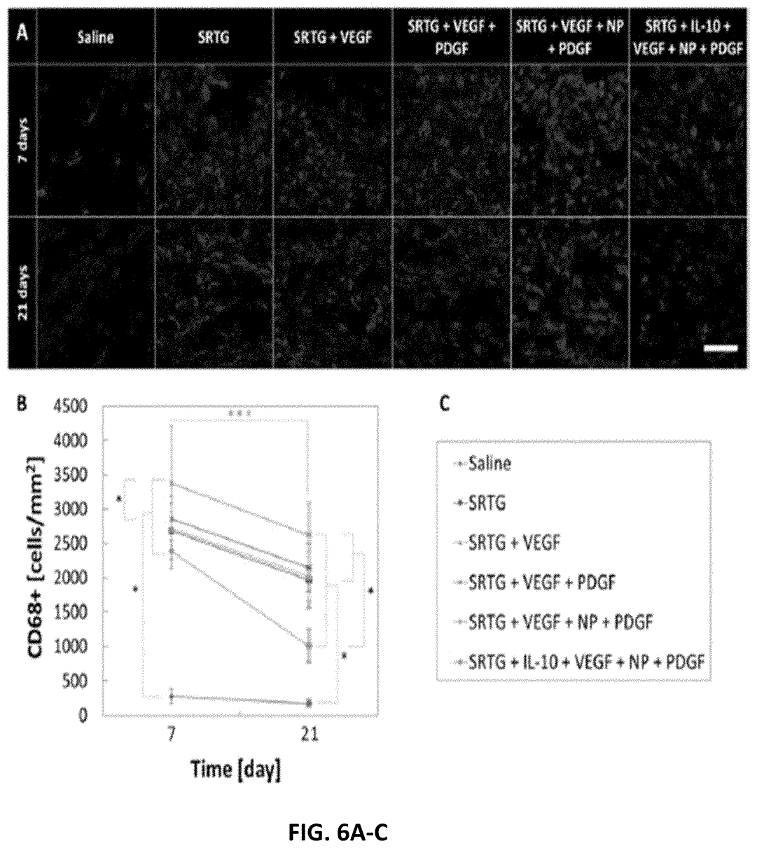

As an injectable delivery system, it is critical that the polymer material is biocompatible. Previously, studies have shown that SRTG does not have cytotoxic properties with a multitude of cells and tissues.^{5, 9, 31} While there is normally an inflammatory reaction caused by foreign materials that are implanted into living tissue, it is imperative that this immune response decreases over time as the material degrades. In this study, immunohistochemistry was used to identify if there was a reduced immune response from the polymer delivery system loaded with IL-10 (FIG. 6). CD68, a glycoprotein that binds to low-density lipoprotein expressed on macrophages, was used to stain and quantify macrophages responding to the polymer material and the released anti-inflammatory cytokine.

SRTG+VEGF+NPs+PDGF+IL-10 exhibited a significant reduction in macrophages compared to the other four polymer injection groups after 21 days (FIG. 6B). This result demonstrates that IL-10 is significantly reducing the immune response caused by the polymer gel. Additionally, the SRTG+VEGF, SRTG+VEGF+PDGF, and SRTG+VEGF+NPs+PDGF+IL-10 all showed a significant reduction in macrophages from 7 to 21 days (FIG. 6B). Although the saline group showed significantly less macrophages than all polymer delivery groups (FIG. 6B), it is expected that the macrophage cell count would continue to decrease over time and ultimately reach normal physiological counts.

An injectable protein polymer delivery system that can revascularize heart tissue could dramatically reduce healthcare costs related to CAD, which would also improve the quality of life for millions of people suffering from this disease. The process of treating CAD can involve many different tests and expensive medications, in addition to the almost inevitable open-heart surgery that must be implemented. A standard injectable protein delivery system that can stimulate new blood vessel formation around a blocked artery could allow many people to avoid paying burdensome healthcare bills. Incorporating a supplementary anti-inflammatory protein into this system might also minimize myocardial necrosis and optimize cardiac repair following MI. Additionally, the polymer system may help with reducing the number of heart attacks experienced by patients because the delivery system could be injected before a complete occlusion of a coronary artery occurs, further reducing healthcare costs. Despite advances in polymer delivery systems for CAD treatment, localized and controlled release of proteins to revascularize damaged heart tissue and reduce the detrimental effects of a prolonged inflammatory response remains inadequate.

The clinical efficacy of angiogenic drug delivery for treating CAD is limited by the formation of unstable neovessels and immature vasculature as a result of the rapid diffusion, poor bioavailability, and short half-lives of angiogenic proteins.⁴² When delivered intravenously, VEGF has a half-life of just over 30 min and is rapidly cleared from the bloodstream.⁴³ However, growth factors that are physically entrapped within heparin-functionalized hydrogels have been shown to increase vascularization and blood flow to the ischemic tissue in vivo,^{44, 45} facilitated through the electrostatic stabilization of the growth factors with heparin, which provides a sustained release of the protein.^{46, 47} Furthermore, several studies have demonstrated the efficacy and importance of dual protein delivery to provide sequential release of angiogenic factors to improve revascularization and heart function after MI, by utilizing a combination of NPs and hydrogels to promote therapeutic angiogenesis.^{15, 20, 42} If angiogenic proteins are delivered simultaneously, early-stage angiogenic factors can interfere with late-stage factors, which can cause immature blood vessel formation.²⁰ The optimal delivery system to improve revascularization and heart function after MI will need to provide sequential, sustained, and local release of growth factors to the damaged heart tissue.

The SRTG delivery system provided spatiotemporal controlled release of VEGF to answer some of these growth factor delivery requirements.^{5, 12} To further improve the SRTG system for treating CAD, novel PDGF-loaded micelle NPs were incorporated, which are embedded within the thermal gel and provide the sequential release of the late-stage angiogenic factor that is needed to stabilize newly formed vessels with vascular smooth muscle cells. The inventors previously demonstrated the release of a drug (triamcinolone acetonide or TA) from polymeric micelles fabricated from a similar triblock copolymer. In that study, the authors examined the release of TA from the micelles alone and from the micelles encapsulated within a similar reverse thermal gel. That release test showed a comparable drug release rate from the encapsulated micelles to the PDGF-loaded micelles shown here, demonstrating that encapsulating the micelles in a reserve thermal gel results in no burst release as seen with the micelles alone.³⁴ Although the inventors have not performed a release study with the PDGF-loaded micelles alone, the inventors believe that the release rate would be analogous to the release profile from the previous study. Based on the triblock polymer structure, there is no functional group with covalent bonding to the drug or protein, hence the inventors believe that there will be a zero-order release rate regardless of which micelle is evaluated.

The polymer delivery system was evaluated for the controlled and sequential release of three proteins using an in vitro release study. The results demonstrated that the SRTG system sequentially released all three factors: VEGF first, followed by IL-10, and finally PDGF. The inventors believe the different heparin-binding affinities of IL-10 and VEGF provided the different release rates between the two factors, although additional testing is necessary to confirm this. Kinetic studies have demonstrated that some angiogenic factors bind to sulfated scaffolds in order of their specific binding affinities (KA);^{37, 48} thus, the inventors believe the proteins are being released with a similar mechanism. Moreover, the release test results showed that PDGF was being slowly released from the micelles that were embedded within the SRTG, which provided the sequential release of PDGF from the scaffold last. This spatiotemporal release of growth factors from the polymer scaffold is critical to promote stable and mature angiogenic vessel formation.

The inventors have provided a modified release profile (FIG. 7), which demonstrates the sustained and sequential release rate of the proteins. The graph was created by removing the burst release and starting the release profile for each factor with the data from day 1, while normalizing the release profile to start at 0. The inventors believe that this may better exemplify the data since it can be difficult to discern the release rates from the full graph. While there are only partial nanogram amounts of each protein releasing after a certain amount of time, the inventors believe that these small amounts are still enough to stimulate a cellular angiogenesis response as demonstrated by the subcutaneous injection study and by evidence from other studies.^{16, 48-52} Additionally, one study reported that extremely low concentrations of TGF-β1, such as 10 pg/mL, stimulated the growth rate of NIH3T3 cells in low serum or serum-free medium.⁵³ With this study taken in conjunction with the animal study, the inventors believe that partial nanogram amounts of growth factor can have a significant cell impact.

PNIPAM-based reverse thermal gels have different properties below and above their respective lower critical solution temperature (LCST, 32° C. for PNIPAM). Lower than the LCST, the polymers are soluble, and the hydrophilic interactions are dominant. Higher than the LCST, the polymers become progressively hydrophobic and insoluble, creating the formation of a physical gel. This transition above the LCST causes the thermoresponsive block to shrink, which causes the proteins to be expelled until the gel has stabilized.⁵⁴ The inventors believe that this is why there is a rapid release of the proteins in the initial first day. The previous study determined that the SRTG goes through a rapid, reversible phase transition around 32° C. (LCST) by UV-visible spectroscopy.³¹ The inventors speculate that the additional heating to body temperature (37° C.) may cause the hydrogels to shrink slightly, causing the proteins to exhibit an initial burst release, until the gel stabilizes. Although there was a burst release for VEGF and IL-10, the inventors see a sustained release over time for each protein after the first day.

Subcutaneous injections of the SRTG polymer system with four different combinations of factors were evaluated for angiogenesis activity and reducing an inflammatory response against saline and SRTG injections. The sequential delivery of VEGF, followed by PDGF, represents the physiological signaling pathway utilized in angiogenesis as the initial vessel formation is activated by VEGF, while vessels are subsequently stabilized by PDGF-facilitated pericyte and smooth muscle cell recruitment. In agreement with the model of sequential growth factor delivery, the SRTG+VEGF+NPs+PDGF+IL-10 group demonstrated the recruitment of significantly more functional vascular endothelial cells and pericytes than every other group after 21 days, except for the SRTG+VEGF+NPs+PDGF group, as expected. This group showed significantly less macrophages present in the subcutaneous tissue compared to all other polymer groups as well. These results show that the polymer delivery system with all three proteins is inducing a substantial angiogenic response with stable and more mature vessels, while reducing inflammation caused by the foreign body response. These results are encouraging as the sequential release of these proteins, in combination with limiting an immune response, can provide optimal cardiac tissue protection following MI. This hypothesis will be examined further when future animal studies are performed using an MI injury mouse model.

The primary goal for this embodiment is toward injecting it into the heart to help optimize cardiac repair and limit the damaging effects of inflammation following MI. The inventors have previously demonstrated that the SRTG scaffold can effectively deliver VEGF to the heart in a MI model to provide cardioprotective benefits.¹² As this system has now demonstrated the ability to deliver multiple angiogenic factors, the inventors are investigating the efficacy of this scaffold for delivering various combinations of heparin-binding proteins, such as basic fibroblast growth factor (FGF-2), stromal cell-derived factor 1 α (SDF-1α), transforming growth factor-β1 (TGF-β1), and hepatocyte growth factor (HGF). The ability to deliver different combinations of these proteins in a localized and spatiotemporal manner could lead to improving left ventricular function after myocardial injury in rodent models.

### Conclusion

Applications of this system could be extended to include other combinations of heparin-binding proteins that are involved in angiogenesis and that act to promote cardiac tissue regeneration. Determining the best synergistic combination of proteins, and the corresponding optimal loading concentrations for each factor needs further investigation. After additional optimization, this spatiotemporal delivery system warrants examination in an MI animal model to determine its capability for protecting cardiac function.

### Example 4—Lidocaine-Loaded PEG-PSHU-PEG Micelles in Crosslinked Hydrogel

Materials and Methods

Micelle Nanoparticle Polymer Synthesis: PEG-PSHU-PEG

NBOC serinol (0.2873 g, 1.5 mmol) and urea (0.09 g, 1.5 mmol) were weighed out and lyophilized at −45° C. and 0.045 mbar for 48 h. The reactants were dissolved in 1.5 mL of anhydrous DMF in a 25 mL round-bottom flask at 90° C. under gentle stirring and a nitrogen atmosphere. HDI (0.482 mL, 3 mmol) was added dropwise, and the polymerization was carried out for 5 days. After the specified time, an excess of poly(ethylene glycol) (PEG 1000, 4 mmol) was dehydrated and added to the reaction. The PEGylation reaction was carried out for 24 h at 90° C. The resulting product, poly(ethylene glycol)-block-poly(serinol hexamethylene urea)-block-poly(ethylene glycol) (PEG-PSHU-PEG), was purified by three precipitations in diethyl ether and then dried completely by extended rotary evaporation at 50° C. and 10 mbar vacuum. Subsequently, the polymer was dissolved in Milli-Q water and dialyzed (MWCO: 3.5 kDa) against 1 L of Milli-Q water for 72 h at room temperature. Then, the product was lyophilized at −45° C. for 48 h to yield a white flaky material.

Micelle Fabrication

Lidocaine-loaded micelles were fabricated by a traditional emulsification-sonication procedure. The PEG-PSHU-PEG polymer and lidocaine (about 1-20 mg polymer to about 50-200 mg lidocaine) were dissolved in 1 mL of DMSO at 3 wt % (polymer/DMSO). This solution was then added dropwise to a beaker containing 20 mL of ultrapure water (milliQ or equivalent) to give a 1:20 organic:aqueous phase ratio partially submerged in an ultrasonic bath. The resulting emulsion was sonicated for 10 min. The removal of DMSO was carried out by centrifugation at 11,000 revolutions per minute (rpm) for 5 min, pouring off the supernatant, and then resuspending the micelles in ultrapure water. This DMSO extraction procedure was carried out three times. The resulting micelles were either used immediately or stored at −20° C. for later use.

Crosslinked Hydrogel Formation—Gelatin and Glutaraldehyde

An aqueous solution containing gelatin was prepared in PBS (5% w/v) and allowed to dissolve overnight in a 37° C. water bath. Micelle nanoparticles loaded with lidocaine-HCl (50-200 mg lidocaine, 1-20 mg polymer) were added to the gelatin solution (0.5 ml gelatin solution). GA was then added to the gelatin-nanoparticles-lidocaine mixture (1% w/v GA) to crosslink the gelatin into a hydrogel.

The delivery system can have applicability to reducing or eliminating pain associated with a wound by being administered topically or injected subcutaneously into a patient in need thereof. The dual micelle-hydrogel system allows for controlled sustained release of an anesthetic such as lidocaine. A further use for the delivery system can be found in Example 6 in which the lidocaine-loaded nanoparticle gel is coated onto the surface of a chest tube to reduce pain associated with placement of the chest tube.

While this embodiment describes lidocaine as the anesthetic, other anesthetics are contemplated for use in the invention.

### Example 5—Lidocaine-Loaded PEG-PSHU-PEG Micelles in Sulfonated Reverse Thermal Gel

Materials and Methods

Synthesis of Sulfonated Reversible Thermal Gel: SRTG

NBOC serinol was synthesized as described previously.⁸ Briefly, serinol and di-tert-butyl dicarbonate were dissolved in ethanol at 4° C. The solution was heated at 37° C. for 1 h, rotoevaporated, and dissolved in an equal volume mixture of ethyl acetate and hexane at 60° C. Additional hexane was added to form crystalline structures, and the precipitate was filtered to remove solvent, yielding N—BOC serinol as a crystalline white product.

PNIPAM was synthesized as described previously.³⁰ In short, NIPAM and 4,4-azobis(4-cyanovaleric acid) were dissolved in methanol and heated at 68° C. for 3 h. PNIPAM was recovered by precipitation in ultrapure water at 60° C., purified via dialysis (MWCO 12-14 kDa), and lyophilized, yielding a white product.

SRTG (SPSHU-PNIPAM) was synthesized similarly as described previously.³¹ N—BOC serinol, urea, and HDI were dissolved in DMF and heated at 90° C. for 7 days. PSHU was recovered by precipitation in diethyl ether and rotoevaporation, yielding the polyurea as a white powder. PSHU was dissolved in DCM and TFA. Deprotection occurred by hydrogenation at room temperature for 45 min providing, free amine groups. Deprotected PSHU (dPSHU) was recovered by precipitation in diethyl ether and rotoevaporation. Further purification of dPSHU involved dissolving in 2,2,2-trifluoroethanol, precipitation in diethyl ether, and rotoevaporation. Next, an equivalent mass of PNIPAM was conjugated to dPSHU. PNIPAM, EDC, and NHS were dissolved in DMF and activated for 24 h. Separately, dPSHU was dissolved in DMF, added to the activated PNIPAM solution, and reacted for 24 h. PSHU-PNIPAM was recovered by precipitation in diethyl ether, rotoevaporation, purified via dialysis (MWCO 12-14 kDa), and lyophilized. Afterward, the remaining free amine groups were sulfonated. 1,3-Propane sultone, potassium tert-butoxide, and PSHU-PNIPAM were dissolved in DMF and reacted at 60° C. for 3 days. SRTG was recovered by precipitation in diethyl ether, rotoevaporation, purified via dialysis (MWCO 12-14 kDa), and lyophilized, yielding a light-yellow product. Polymer characterization was performed as described previously to confirm material produced.^{5, 9, 32}

Micelle Nanoparticle Polymer Synthesis: PEG-PSHU-PEG

NBOC serinol (0.2873 g, 1.5 mmol) and urea (0.09 g, 1.5 mmol) were weighed out and lyophilized at −45° C. for 48 h. The reactants were dissolved in 1.5 mL of anhydrous DMF in a 25 mL round-bottom flask at 90° C. under gentle stirring and a nitrogen atmosphere. HDI (0.482 mL, 3 mmol) was added dropwise, and the polymerization was carried out for 5 days. After the specified time, an excess of poly(ethylene glycol) (PEG 1000, 4 mmol) was dehydrated and added to the reaction. The PEGylation reaction was carried out for 24 h at 90° C. The resulting product, poly(ethylene glycol)-blockpoly(serinol hexamethylene urea)-block-poly(ethylene glycol) (PEGPSHU-PEG), was purified by three precipitations in diethyl ether and then dried completely by extended rotary evaporation at 50° C. and 10 mbar vacuum. Subsequently, the polymer was dissolved in Milli-Q water and dialyzed (MWCO: 3.5 kDa) against 1 L of Milli-Q water for 72 h at room temperature. Then, the product was lyophilized at −45° C. for 48 h to yield a white flaky material.

Micelle Fabrication

Lidocaine-loaded micelles were fabricated by a traditional emulsification-sonication procedure. The PEG-PSHU-PEG polymer and lidocaine were dissolved in 1 mL of DMSO at 3 wt % (polymer/DMSO). This solution was then added dropwise to a beaker containing 20 mL of ultrapure water partially submerged in an ultrasonic bath. The resulting emulsion was sonicated for 10 min. The removal of DMSO was carried out by centrifugation at 11,000 revolutions per minute (rpm) for 5 min, pouring off the supernatant, and then resuspending the micelles in ultrapure water. This DMSO extraction procedure was carried out three times. The resulting micelles were either used immediately or stored at −20° C. for later use.

### Example 6—Lidocaine Loaded PEG-PSHU-PEG Micelles Embedded in Crosslinked Gelatin Used to Coat Chest Tube

Several studies have examined techniques to minimize pain with chest drains including epidurals, peri-vertebral catheters, rib blocks, pleural infusions of bupivacaine and application of lidocaine jelly to chest tubes.⁶⁰⁻⁶² These techniques have not been widely adopted due to inconsistent results, undesirable hemodynamic side effects, expense, requirement for specialized equipment, required teaching for staff, or significant modification of intraoperative practice.

Prospective, randomized studies have tested the efficacy of using the lumen of the chest tube or a separate intrathoracic catheter to deliver an infusion or bolus of intrathoracic bupivacaine after thoracotomy or video assisted thoracoscopy with mixed results.^{60, 61} One single-center, prospective, randomized study applied 2% lidocaine jelly on chest tubes placed after CABG and reported a statistically significant decrease in both reported pain and opioid requirement for pain control in the immediate post-operative period.⁶² However, there are several challenges to the broad acceptance of this technique: 1) concern that the jelly would slide off the chest tube as it is tunneled through the skin; 2) concern for occlusion of drain channels, obstructing drainage of air or blood which is clinically unacceptable; 3) concern about inherent variability in application of jelly and absorption of lidocaine; and 4) with no controlled-release of lidocaine, the duration of this analgesia is short-lived.

In order to overcome these limitations, the inventors have developed lidocaine loaded micelle nanoparticles which can be embedded within a bio-absorbable polymer hydrogel to form a drug delivery system that can be applied to a catheter, such as a chest tube, to allow sustained release of the lidocaine over a period of between about 3-7 days. Polymer concentration, lidocaine-loading amount and crosslinker concentration can be optimized to control the amount and rate of lidocaine release.

Materials and Methods

Lidocaine-loaded PEG-PSHU-PEG micelles were manufactured as described previously in Example 4 and detailed again below.

Micelle Nanoparticle Polymer Synthesis: PEG-PSHU-PEG

NBOC serinol (0.2873 g, 1.5 mmol) and urea (0.09 g, 1.5 mmol) were weighed out and lyophilized at −45° C. and 0.045 mbar for 48 h. The reactants were dissolved in 1.5 mL of anhydrous DMF in a 25 mL round-bottom flask at 90° C. under gentle stirring and a nitrogen atmosphere. HDI (0.482 mL, 3 mmol) was added dropwise, and the polymerization was carried out for 5 days. After the specified time, an excess of poly(ethylene glycol) (PEG 1000, 4 mmol) was dehydrated and added to the reaction. The PEGylation reaction was carried out for 24 h at 90° C. The resulting product, poly(ethylene glycol)-block-poly(serinol hexamethylene urea)-block-poly(ethylene glycol) (PEG-PSHU-PEG), was purified by three precipitations in diethyl ether and then dried completely by extended rotary evaporation at 50° C. and 10 mbar vacuum. Subsequently, the polymer was dissolved in Milli-Q water and dialyzed (MWCO: 3.5 kDa) against 1 L of Milli-Q water for 72 h at room temperature. Then, the product was lyophilized at −45° C. for 48 h to yield a white flaky material.

Micelle Fabrication

Lidocaine-loaded micelles were fabricated by a traditional emulsification-sonication procedure. The PEG-PSHU-PEG polymer and lidocaine (about 1-20 mg polymer to about 50-200 mg lidocaine) were dissolved in 1 mL of DMSO at 3 wt % (polymer/DMSO). This solution was then added dropwise to a beaker containing 20 mL of ultrapure water (milliQ or equivalent) to give a 1:20 organic:aqueous phase ratio partially submerged in an ultrasonic bath. The resulting emulsion was sonicated for 10 min. The removal of DMSO was carried out by centrifugation at 11,000 revolutions per minute (rpm) for 5 min, pouring off the supernatant, and then resuspending the micelles in ultrapure water. This DMSO extraction procedure was carried out three times. The resulting micelles were either used immediately or stored at −20° C. for later use.

Crosslinked Hydrogel Formation—Gelatin and Glutaraldehyde

An aqueous solution containing gelatin was prepared in PBS (5% w/v) and allowed to dissolve overnight in a 37° C. water bath. Micelle nanoparticles loaded with lidocaine-HCl (50-200 mg lidocaine, 1-20 mg polymer) were added to the gelatin solution (0.5 ml gelatin solution). GA was then added to the gelatin-nanoparticles-lidocaine mixture (1% w/v GA) to crosslink the gelatin into a hydrogel.

Application of Micelle-Loaded Crosslinked Hydrogel onto Chest Tube

The crosslinked hydrogel containing the embedded lidocaine-loaded micelles was applied to the chest tube and dehydrated thus resulting in a thin, stable polymer coating on the tubing. For the application of the hydrogel to the tubing, before the hydrogel was fully formed, the viscous solution was poured onto the catheter tubing and allowed to fully polymerize for 5 minutes. The tubing was slowly spun around by hand during the polymerization, to ensure a smooth, even coating of the gel. From here, the hydrogel coating on the tubing can be fully dehydrated overnight at room temperature or used immediately for further applications. The fully dehydrated samples result in a thin, rigid polymer coating on the tubing, while the fresh hydrogel samples produce a thick, elastic, jelly-like coating. Both forms of the gelatin coating were analyzed with an ex vivo rib test and a lidocaine release study was performed.

Additionally, a mold can be used, instead of hand-spinning, to apply the hydrogel to the tubing. For mold application, an aqueous solution containing gelatin was prepared in PBS (5% w/v) and allowed to dissolve overnight in a 37° C. water bath. Lidocaine-HCl was dissolved in the gelatin solution (200 mg lidocaine/1 ml gelatin solution). Glutaraldehyde was then added to the gelatin-lidocaine mixture (1% w/v) to crosslink the gelatin into a hydrogel. Before the hydrogel was fully formed, the tubing was inserted into the mold containing the gelatin-glutaraldehyde solution and the polymerization was allowed to continue until fully gelled (approximately 1 hour). Subsequently, the hydrogel coated tubing was removed from the mold. After polymerization the coated chest tubes are dehydrated to form a stably fixed coating. The introduction of a lyophilizer may control the dehydration step and further aid in eliminating variation in elution that may be related to this step.

In order to prevent obstruction of any orifices or channels, a nasogastric tube was inserted into the chest tube lumen during the coating and polymerization process. For a Blake drain, wooden skewers were inserted into the lumen allowing the entire length of the Blake drain to be coated.

Sterilization of Coated Tube

The coating may be applied in the operating room similar to the application of BioGlue®. Applying the coating in the operating room has the advantage of being able to adjust the dose if there is a concern for toxicity based on weight or hepatic impairment. Alternatively, components can be filtered prior to crosslinking, applied to the tube and the tube subsequently packaged in a sealed bag and sterilized in an autoclave for in vivo application.

Ex Vivo Rib Test

To determine whether coating the chest tube with the micelle-loaded crosslinked hydrogel composition would be durable and stable an exemplary chest tube is coated as described above with the composition. The coated chest tube is then inserted ex vivo between pork spareribs using a technique simulating chest tube insertion in a patient and the durability and stability of the coating is observed.

Scanning Electron Microscopy (SEM) Analysis

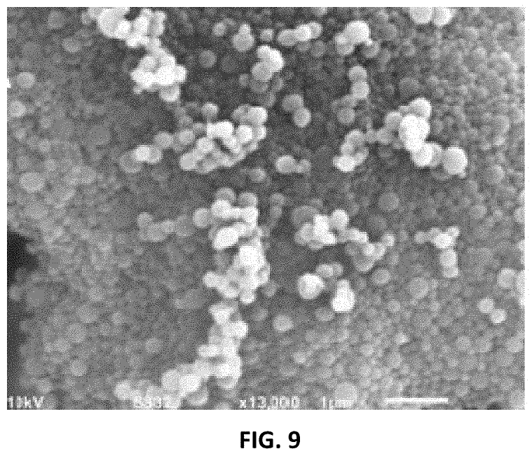

To examine polymer morphology on the nanometer scale, SEM is performed for the lidocaine-polymer coated chest tube before and after autoclave sterilization. (FIG. 9)

Lidocaine Release Study

For the lidocaine release study, a coated tubing which comprises a dried 1 cm by 0.5 cm piece of gelatin-lidocaine (100 mg lidocaine in 0.5 ml gelatin, 1% GA) was used. The coated tubing was placed in a 7 ml glass scintillation vial for the release study. The release solution medium (1 ml of PBS) was added to the scintillation vials, and the sample was incubated at 37° C. 1 ml samples were taken daily, and the release solution was replenished. The day 0 sample was taken 5 minutes after adding the release solution to the vials to measure the amount of lidocaine not incorporated into the gels.

The amount of lidocaine released as an effect of different nanoparticle formulations and differing amounts of lidocaine was determined using a UV-Vis spectrophotometer (GENESYS 10S UV-Vis) at 280 nm wavelength and samples were performed in triplicate (n=3). As a control, the same gelatin samples were prepared without the addition of lidocaine to measure any change in absorbance readings from the gel itself. Absorbance readings from the control samples were subtracted from the experimental lidocaine samples to remove any background polymer that may have interfered with the quantification of released lidocaine.

In Vivo Testing

To control for the pain tolerance of an individual subject (Sprague Dawley rats and later pigs), one lidocaine coated piece of tube is inserted subcutaneously over the posterior rib cage on one side and an uncoated piece of tube in the exact location on the opposite side. Power analysis using the resource equation estimates 22 subjects to detect a difference of 20% (nociceptive pain scale 0-10). Using a single subject to evaluate both the control and experimental tube, 11 subjects are needed. Additional controls include lidocaine-specific morbidity (see group 4), Von Frey testing at baseline (see group 1) and the incision alone (see group 2).

In Vivo Prototype Preparation

5 mm×10 mm chest tube pieces are coated with equal volumes of lidocaine-polymer. SEM and lidocaine release testing are performed before and after autoclave sterilization.

In Vivo Testing Experimental Design

An IACUC approved protocol (12) is used to perform in vivo testing. Group 1: No incision or chest tube (2 subjects); Group 2: Bilateral incisions, no chest tube (2 subjects); Group 3: One control tube, one lidocaine-polymer coated tube (11 subjects); Group 4: One Lidocaine-polymer tube only (2 subjects).

Data collected include daily weight, food consumption, mobility, analgesia requirements, other morbidity and mortality.^{63, 64} Drug levels and inflammatory markers are measured daily out to 7 days.

Pain Testing Using Von Frey Fibers:

Behavior and in vivo neurophysiology core technicians perform daily, blinded, non-biased assessment of pain. The skin is probed with Von Frey hairs around the tube and response graded on the nociceptive scale. How far from the tube a change in nociception occurs is also assessed.

Histology

Tissue is prepared for histology. Core services are utilized to characterize scar formation, cytotoxicity and inflammation.

Results

Synthesis of PEG-PSHU-PEG and Micelle Fabrication and Characterization

Novel micelle NPs were created from a PEG-PSHU-PEG polymer. The PSHU backbone provides the hydrophobic core of the micelles, while the terminal PEG chains provide the hydrophilic interactions on the exterior shell. PEG adds an additional benefit as this provides a “stealth” effect to the NPs for an extended half-life in circulation. Scanning electron microscopy (SEM) was used to characterize the morphologies of the micelles. The micelle NPs were uniformly spherical (FIG. 9).

Release Rates of Different Nanoparticle Formulations

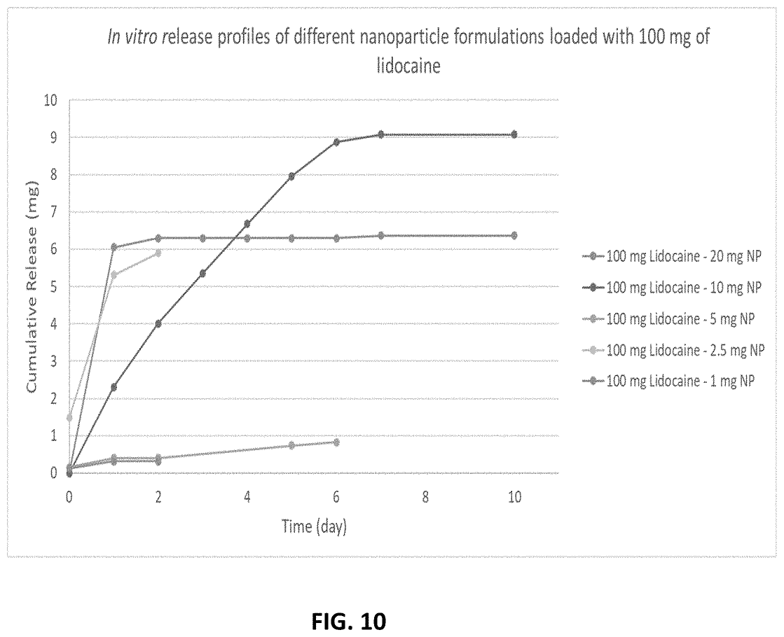

As shown in FIG. 10, the release profiles from the various NP formulations show the differences in lidocaine release rates are dependent on the amount of NP loaded into the hydrogels. The data display the sustained release of the lidocaine over 10 days for the 20 mg and 10 mg NP groups. The data for last day of each group shows last timepoint where lidocaine could be detected in the release solution.

Release Rates for Different Nanoparticle Formulations Having Differing Amounts of Lidocaine

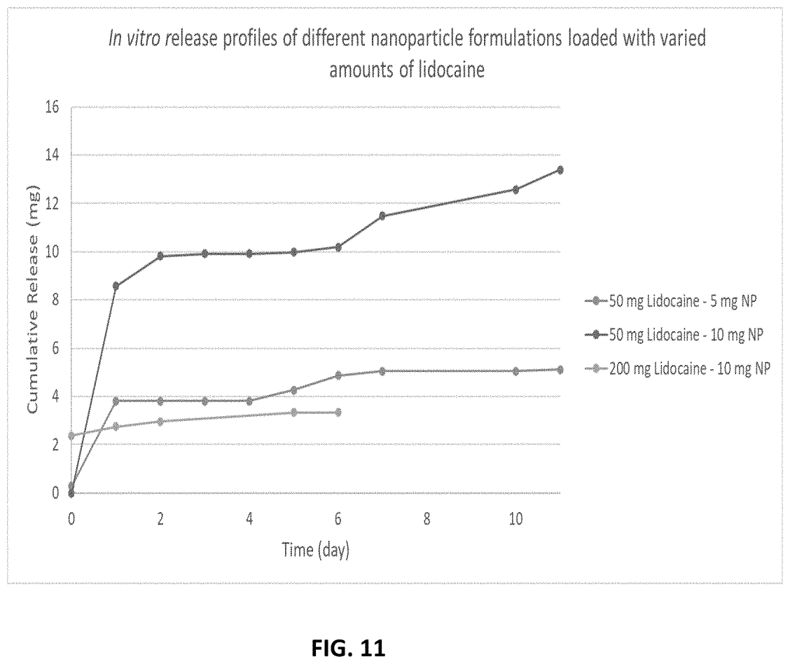

FIG. 11 depicts the in vitro profiles of different nanoparticle formulations that have been loaded with varied amounts of lidocaine. The release profiles from the various NP formulations show the differences in lidocaine release rates are dependent on the amount of NP and lidocaine loaded into the hydrogels. The data display the sustained release of the lidocaine over 11 days for the 50 mg lidocaine groups. The data for last day of each group shows last timepoint where lidocaine could be detected in the release solution.

Optimal Gelatin Hydrogel Solution Formulations with Embedded Nanoparticles

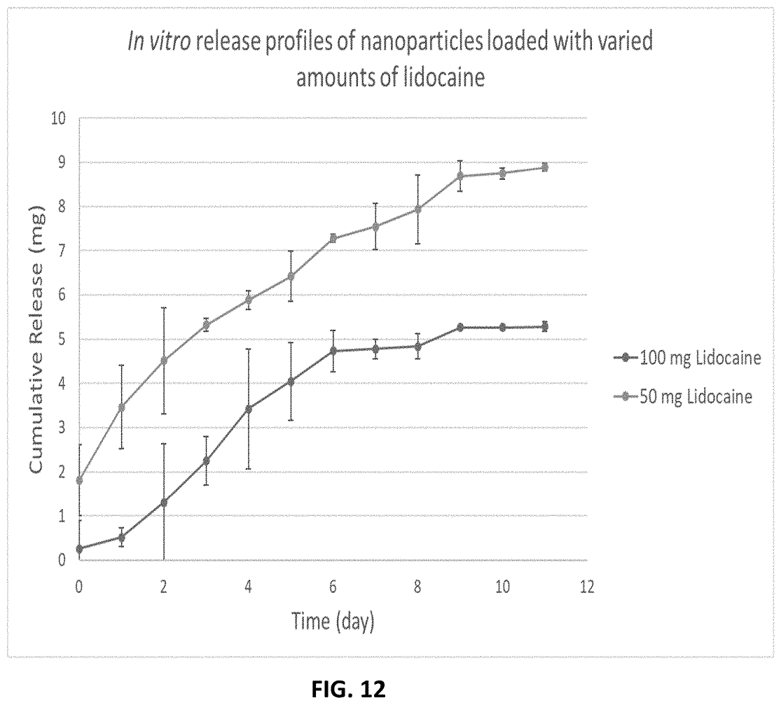

Based on the release data shown previously, the optimal formulations of the gelatin-nanoparticle delivery system were selected for additional examination with release tests using three samples each. Embedding 10 mg of nanoparticles into the hydrogel, while loading 50 or 100 mg of lidocaine in the nanoparticles, displayed the ideal sustained release profile of lidocaine over 11 days. These two formulations were further examined to additional release testing. The release tests show the sustained and linear release of lidocaine from the hydrogel delivery system with either 100 or 50 mg of the drug loaded into the nanoparticles (FIG. 12).

Subcutaneous Implantation Mouse Model of Catheter Tubing with and without Gelatin-Nanoparticle Hydrogel Coating

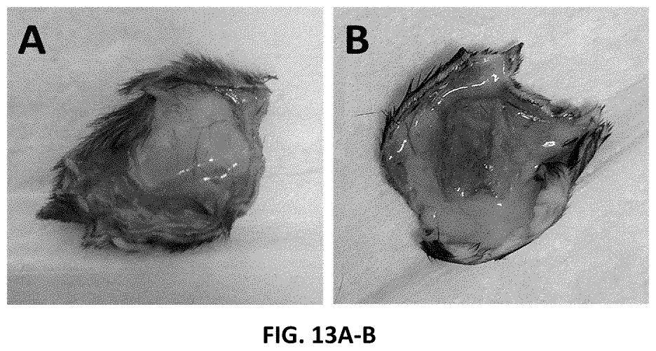

Gelatin-nanoparticle hydrogel coated tubing samples were prepared as described previously. Nanoparticles (0.2 mg) were loaded with 1 mg of lidocaine and embedded within gelatin hydrogels for coating procedure using a 0.5 cm by 1 cm piece of catheter tubing. For the mouse model, a 2 cm incision was made in the skin over the right posterior chest and a subcutaneous space was created. The silicone tube was inserted either uncoated or coated depending on whether control or experimental. The entirety of the tube was implanted subcutaneously, and the incision was closed with an external suture closure. The mice were euthanized on postoperative day 14 after the procedure. Tissue from the skin around the tube was harvested and analyzed by gross examination (FIG. 13). The images show similar fibrous-tissue capsule formation between the coated and uncoated tubing samples.

Lidocaine Loaded Micelles Embedded in Crosslinked Gelatin Hydrogel Produce a Durable and Stable Coating to Chest Tube

The coating does not flake off the chest tube after being applied. The coating moves in coordination with the tube thus allowing the tube to remain flexible. When inserted between the ribs in the ex vivo test the coating remains stably fixed on the chest tube. The stable fixation of the coating allows the surgeon to maintain their techniques for handling and placing the chest tube in the patient.

In addition, the stable fixation of the coating to the tube prevents obstruction of the chest tube openings or central lumen. Similarly, when using a Blake drain, none of the drain channels are blocked.

Coated Chest Tube is Able to Stably Release Lidocaine

A post-sterilization lidocaine release profile indicates continued stable elution over a period of about 3-7 days. The maximum subcutaneous dose of lidocaine hydrochloride is about 4.5 mg/kg with a maximum dose at one time not to exceed 300 mg.⁶⁷ The targeted lidocaine release for a chest tube or Blake drain is 4.5 mg/kg/day.

While this embodiment describes lidocaine as the anesthetic, other anesthetics are contemplated for use in the invention. Similarly, while a chest tube is used in this embodiment, other types of catheters may be similarly coated.

### Example 7—Lidocaine Loaded Crosslinked Gelatin Used to Coat Chest Tube

Gelatin-Lidocaine Hydrogel Formulation

An aqueous solution containing gelatin was prepared in PBS (5% w/v or 10% w/v) and allowed to dissolve overnight in a 37° C. water bath. Lidocaine-HCl was dissolved in the gelatin solution (200 mg lidocaine/1 ml gelatin solution). Glutaraldehyde (GA) was then added to the gelatin-lidocaine mixture (1-4% w/v) to crosslink the gelatin into a hydrogel. Before the hydrogel was fully formed, the viscous solution was poured onto the catheter tubing and allowed to fully polymerize for 5 minutes. The tubing was slowly spun around by hand during the polymerization, to ensure a smooth, even coating of the gel. From here, the hydrogel coating on the tubing can be fully dehydrated overnight at room temperature or used immediately for further applications. The fully dehydrated samples result in a thin, rigid polymer coating on the tubing, while the fresh hydrogel samples produce a thick, elastic, jelly-like coating. Both forms of the gelatin coating were analyzed with an ex vivo rib test and a lidocaine release study was performed as described below.

In some embodiments, the introduction of a lyophilizer may control the dehydration step and further aid in eliminating variation in elution that may be related to this step.

In order to prevent obstruction of any orifices or channels, a nasogastric tube was inserted into the chest tube lumen during the coating and polymerization process. For a Blake drain, wooden skewers were inserted into the lumen allowing the entire length of the Blake drain to be coated.

Sterilization of Coated Tube

The coating may be applied in the operating room similar to the application of BioGlue®. Applying the coating in the operating room has the advantage of being able to adjust the dose if there is a concern for toxicity based on weight or hepatic impairment. Alternatively, components can be filtered prior to crosslinking, applied to the tube and the tube subsequently packaged in a sealed bag and sterilized in an autoclave for in vivo application.

Ex Vivo Rib Test

To determine whether coating the chest tube with the micelle-loaded crosslinked hydrogel composition would be durable and stable an exemplary chest tube is coated as described above with the composition. The coated chest tube is then inserted ex vivo between pork spareribs using a technique simulating chest tube insertion in a patient and the durability and stability of the coating is observed.

Lidocaine Release Study

For the lidocaine release study, two methods were used to assess the potential drug release difference between the hydrated and dehydrated coatings. The first method uses a coated tubing which comprises a dried 1 cm by 0.5 cm piece of gelatin-lidocaine (100 mg lidocaine in 0.5 ml gelatin, 1% GA). The coated tubing was placed in a 7 ml glass scintillation vial for the release study. The second method uses a gel mold comprising a fresh, hydrated gelatin-lidocaine gel (200 mg lidocaine in 1 ml gelatin, 1% GA) that was polymerized directly into a 7 ml glass scintillation vial thus forming a hydrogel disc. The hydrogel disc was allowed to stabilize for 30 minutes before examining the lidocaine release.

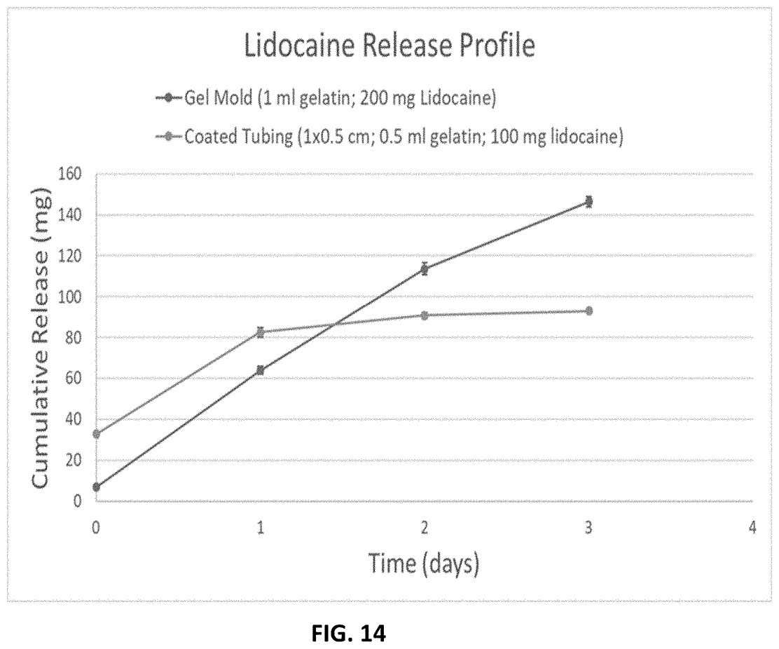

The release solution medium (1 ml of PBS) was added to the scintillation vials, and the sample was incubated at 37° C. 1 ml samples were taken daily, and the release solution was replenished. The day 0 sample was taken 5 minutes after adding the release solution to the vials to measure the amount of lidocaine not incorporated into the gels. The amount of lidocaine released was determined using a UV-Vis spectrophotometer (GENESYS 10S UV-Vis) at 280 nm wavelength and samples were performed in triplicate (n=3) as shown in FIG. 14. As a control, the same gelatin samples were prepared without the addition of lidocaine to measure any change in absorbance readings from the gel itself. Absorbance readings from the control samples were subtracted from the experimental lidocaine samples to remove any background polymer that may have interfered with the quantification of released lidocaine.

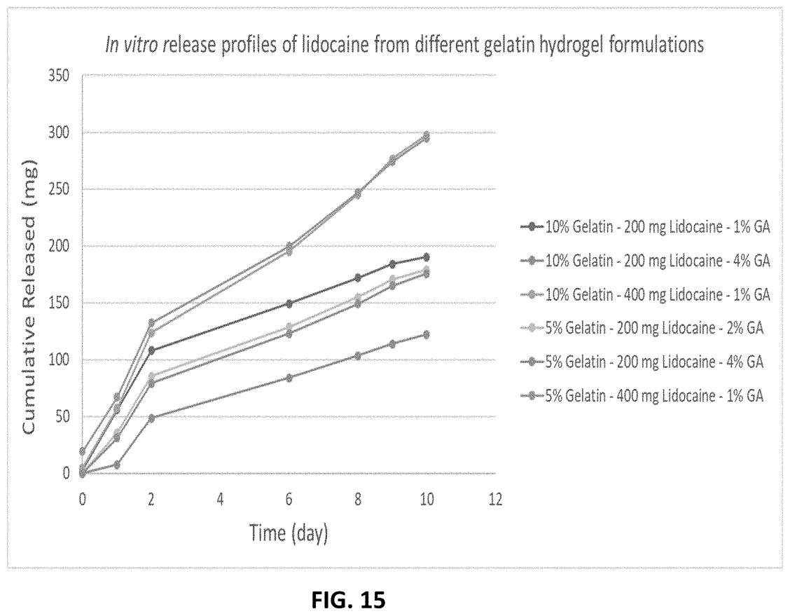

Additional release studies were performed to examine the effects of changing gelatin and GA concentrations on the release rate of lidocaine (FIG. 15). For this study, all samples were prepared using the coated tubing method to demonstrate clinically translational feasibility of this delivery system. The amount of lidocaine released was determined using the same UV-Vis spectrophotometer method.

The release profiles from the various gelatin formulations show the differences in lidocaine release rates are dependent on the concentration of gelatin and glutaraldehyde (GA). The data display the sustained release of the lidocaine over 10 days.

Scanning Electron Microscopy (SEM) Analysis

To examine polymer morphology on the nanometer scale, SEM is performed for the lidocaine-polymer coated chest tube before and after autoclave sterilization.

In Vivo Testing

To control for the pain tolerance of an individual subject (Sprague Dawley rats and later pigs), one lidocaine coated piece of tube is inserted subcutaneously over the posterior rib cage on one side and an uncoated piece of tube in the exact location on the opposite side. Power analysis using the resource equation estimates 22 subjects to detect a difference of 20% (nociceptive pain scale 0-10). Using a single subject to evaluate both the control and experimental tube, 11 subjects are needed. Additional controls include lidocaine-specific morbidity (see group 4), Von Frey testing at baseline (see group 1) and the incision alone (see group 2).

In Vivo Prototype Preparation

5 mm×10 mm chest tube pieces are coated with equal volumes of lidocaine-polymer. SEM and lidocaine release testing are performed before and after autoclave sterilization.

In Vivo Testing Experimental Design

An IACUC approved protocol (12) will be used to perform in vivo testing. Group 1: No incision or chest tube (2 subjects); Group 2: Bilateral incisions, no chest tube (2 subjects); Group 3: One control tube, one lidocaine-polymer coated tube (11 subjects); Group 4: One Lidocaine-polymer tube only (2 subjects).

Data collected include daily weight, food consumption, mobility, analgesia requirements, other morbidity and mortality.^{63, 64} Drug levels and inflammatory markers are measured daily out to 7 days.

Pain Testing Using Von Frey Fibers:

Behavior and in vivo neurophysiology core technicians perform daily, blinded, non-biased assessment of pain. The skin is probed with Von Frey hairs around the tube and response graded on the nociceptive scale. How far from the tube a change in nociception occurs is also assessed.

Histology

Tissue is prepared for histology. Core services are utilized to characterize scar formation, cytotoxicity and inflammation.

Results

Lidocaine-Containing Crosslinked Gelatin Hydrogel Produce a Durable and Stable Coating to Chest Tube

The coating does not flake off the chest tube after being applied. The coating moves in coordination with the tube thus allowing the tube to remain flexible. When inserted between the ribs in the ex vivo test the coating remains stably fixed on the chest tube. The stable fixation of the coating allows the surgeon to maintain their techniques for handling and placing the chest tube in the patient.

In addition, the stable fixation of the coating to the tube prevents obstruction of the chest tube openings or central lumen. Similarly, when using a Blake drain, none of the drain channels are blocked.

Coated Chest Tube is Able to Stably Release Lidocaine

A post-sterilization lidocaine release profile indicates continued stable elution over a period of about 3-7 days. The maximum subcutaneous dose of lidocaine hydrochloride is about 4.5 mg/kg with a maximum dose at one time not to exceed 300 mg.⁶⁷ The targeted lidocaine release for a chest tube or Blake drain is 4.5 mg/kg/day.

While this embodiment describes lidocaine as the anesthetic, other anesthetics are contemplated for use in the invention. Similarly, while a chest tube is used in this embodiment, other types of catheters may be similarly coated.

The concept of a drug-eluting drain has significant scientific importance because of its potential use for any drain placed inside the body. In addition, the technique of using a bioabsorbable polymer for drug delivery signifies the potential to deliver different types of drugs or combination of drugs, with elution profiles tailored to the application. Drug delivery on a bio-absorbable scaffold is a rapidly evolving field.

### Example 8—Lidocaine Loaded PEG-PSHU-PEG Micelles Embedded in Lidocaine-Loaded Crosslinked Gelatin Used to Coat Chest Tube—Prophetic

The inventors have developed lidocaine loaded micelle nanoparticles which can be embedded within a lidocaine-loaded bio-absorbable polymer hydrogel to form a drug delivery system that can be applied to a catheter, such as a chest tube, to allow for immediate as well as sustained release of the lidocaine. Polymer concentration, lidocaine-loading amount and crosslinker concentration can be optimized to control the amount and rate of lidocaine release.

Materials and Methods

Lidocaine-loaded PEG-PSHU-PEG micelles were manufactured as described previously and detailed again below.

Micelle Nanoparticle Polymer Synthesis: PEG-PSHU-PEG

NBOC serinol (0.2873 g, 1.5 mmol) and urea (0.09 g, 1.5 mmol) were weighed out and lyophilized at −45° C. for 48 h. The reactants were dissolved in 1.5 mL of anhydrous DMF in a 25 mL round-bottom flask at 90° C. under gentle stirring and a nitrogen atmosphere. HDI (0.482 mL, 3 mmol) was added dropwise, and the polymerization was carried out for 5 days. After the specified time, an excess of poly(ethylene glycol) (PEG 1000, 4 mmol) was dehydrated and added to the reaction. The PEGylation reaction was carried out for 24 h at 90° C. The resulting product, poly(ethylene glycol)-blockpoly(serinol hexamethylene urea)-block-poly(ethylene glycol) (PEGPSHU-PEG), was purified by three precipitations in diethyl ether and then dried completely by extended rotary evaporation at 50° C. and 10 mbar vacuum. Subsequently, the polymer was dissolved in Milli-Q water and dialyzed (MWCO: 3.5 kDa) against 1 L of Milli-Q water for 72 h at room temperature. Then, the product was lyophilized at −45° C. for 48 h to yield a white flaky material.

Micelle Fabrication

Lidocaine-loaded micelles were fabricated by a traditional emulsification-sonication procedure. The PEG-PSHU-PEG polymer and lidocaine were dissolved in 1 mL of DMSO at 3 wt % (polymer/DMSO). This solution was then added dropwise to a beaker containing 20 mL of ultrapure water partially submerged in an ultrasonic bath. The resulting emulsion was sonicated for 10 min. The removal of DMSO was carried out by centrifugation at 11,000 revolutions per minute (rpm) for 5 min, pouring off the supernatant, and then resuspending the micelles in ultrapure water. This DMSO extraction procedure was carried out three times. The resulting micelles were either used immediately or stored at −20° C. for later use.

Gelatin-Lidocaine Crosslinked Hydrogel Formation

An aqueous solution containing gelatin is prepared in PBS (5% w/v or 10% w/v) and allowed to dissolve overnight in a 37° C. water bath. Lidocaine-HCl is dissolved in the gelatin solution (200 mg lidocaine/1 ml gelatin solution). Micelle nanoparticles loaded with lidocaine-HCl (50-200 mg lidocaine, 1-20 mg polymer) are added to the gelatin solution (0.5 ml gelatin solution). Glutaraldehyde (GA) is then added to the gelatin-micelle-lidocaine mixture (1-4% w/v) to crosslink the gelatin into a hydrogel.

Application of Micelle-Loaded Crosslinked Gelatin-Lidocaine Hydrogel onto Chest Tube

The crosslinked hydrogel containing the embedded lidocaine-loaded micelles are applied to the chest tube and dehydrated thus resulting in a thin, stable polymer coating on the tubing. For the application of the hydrogel to the tubing, a hydrogel containing is prepared as described above. Before the hydrogel is fully formed, the viscous solution is poured onto the catheter tubing and allowed to fully polymerize for 5 minutes. The tubing is slowly spun around by hand during the polymerization, to ensure a smooth, even coating of the gel. From here, the hydrogel coating on the tubing can be fully dehydrated overnight at room temperature or used immediately for further applications. The fully dehydrated samples result in a thin, rigid polymer coating on the tubing, while the fresh hydrogel samples produce a thick, elastic, jelly-like coating. Both forms of the gelatin coating are analyzed with an ex vivo rib test and a lidocaine release study is performed.

Additionally, a mold can be used, instead of hand-spinning, to apply the hydrogel to the tubing. For mold application, an aqueous solution containing gelatin is prepared in PBS (5% w/v) and allowed to dissolve overnight in a 37° C. water bath. Lidocaine-HCl was dissolved in the gelatin solution (200 mg lidocaine/1 ml gelatin solution). Micelle nanoparticles loaded with lidocaine-HCl (50-200 mg lidocaine, 1-20 mg polymer) are added to the gelatin solution (0.5 ml gelatin solution). Glutaraldehyde is then added to the gelatin-lidocaine mixture (1% w/v) to crosslink the gelatin into a hydrogel. Before the hydrogel was fully formed, the tubing is inserted into the mold containing the gelatin-micelle-glutaraldehyde solution and the polymerization was allowed to continue until fully gelled (approximately 1 hour). Subsequently, the hydrogel coated tubing was removed from the mold. After polymerization the coated chest tubes are dehydrated to form a stably fixed coating. The introduction of a lyophilizer may control the dehydration step and further aid in eliminating variation in elution that may be related to this step.

In order to prevent obstruction of any orifices or channels, a nasogastric tube is inserted into the chest tube lumen during the coating and polymerization process. For a Blake drain, wooden skewers are inserted into the lumen allowing the entire length of the Blake drain to be coated.

Sterilization of Coated Tube

The coating may be applied in the operating room similar to the application of BioGlue®. Applying the coating in the operating room has the advantage of being able to adjust the dose if there is a concern for toxicity based on weight or hepatic impairment. Alternatively, components can be filtered prior to crosslinking, applied to the tube and the tube subsequently packaged in a sealed bag and sterilized in an autoclave for in vivo application.

Ex Vivo Rib Test

To determine whether coating the chest tube with the micelle-loaded crosslinked hydrogel composition would be durable and stable an exemplary chest tube is coated as described above with the composition. The coated chest tube is then inserted ex vivo between pork spareribs using a technique simulating chest tube insertion in a patient and the durability and stability of the coating is observed.

Lidocaine Release Study

Lidocaine release is determined using a UV-Vis spectrophotometer (GENESYS 10S UV-Vis) at 280 nm wavelength. The coated chest tube is in a sealed vial with phosphate buffered saline (PBS) and aliquots tested every 24 hours. Testing is performed both before and after autoclave sterilization.

Scanning Electron Microscopy (SEM) Analysis

To examine polymer morphology on the nanometer scale, SEM is performed for the lidocaine-polymer coated chest tube before and after autoclave sterilization.

In Vivo Testing

To control for the pain tolerance of an individual subject (Sprague Dawley rats and later pigs), one lidocaine coated piece of tube is inserted subcutaneously over the posterior rib cage on one side and an uncoated piece of tube in the exact location on the opposite side. Power analysis using the resource equation estimates 22 subjects to detect a difference of 20% (nociceptive pain scale 0-10). Using a single subject to evaluate both the control and experimental tube, 11 subjects are needed. Additional controls include lidocaine-specific morbidity (see group 4), Von Frey testing at baseline (see group 1) and the incision alone (see group 2).

In Vivo Prototype Preparation

5 mm×10 mm chest tube pieces are coated with equal volumes of lidocaine-polymer. SEM and lidocaine release testing are performed before and after autoclave sterilization.

In Vivo Testing Experimental Design

An IACUC approved protocol (12) is used to perform in vivo testing. Group 1: No incision or chest tube (2 subjects); Group 2: Bilateral incisions, no chest tube (2 subjects); Group 3: One control tube, one lidocaine-polymer coated tube (11 subjects); Group 4: One Lidocaine-polymer tube only (2 subjects).

Data collected include daily weight, food consumption, mobility, analgesia requirements, other morbidity and mortality.^{63, 64} Drug levels and inflammatory markers are measured daily out to 7 days.

Pain Testing Using Von Frey Fibers:

Behavior and in vivo neurophysiology core technicians perform daily, blinded, non-biased assessment of pain. The skin is probed with Von Frey hairs around the tube and response graded on the nociceptive scale. How far from the tube a change in nociception occurs is also assessed.

Histology

Tissue is prepared for histology. Core services are utilized to characterize scar formation, cytotoxicity and inflammation.

Results

Synthesis of PEG-PSHU-PEG and Micelle Fabrication and Characterization

Novel micelle NPs were created from a PEG-PSHU-PEG polymer. The PSHU backbone provides the hydrophobic core of the micelles, while the terminal PEG chains provide the hydrophilic interactions on the exterior shell. PEG adds an additional benefit as this provides a “stealth” effect to the NPs for an extended half-life in circulation. Scanning electron microscopy (SEM) was used to characterize the morphologies of the micelles. The micelle NPs were uniformly spherical (FIG. 9).

Lidocaine Loaded Micelles Embedded in Crosslinked Gelatin-Lidocaine Hydrogel Produce a Durable and Stable Coating to Chest Tube

The coating does not flake off the chest tube after being applied. The coating moves in coordination with the tube thus allowing the tube to remain flexible. When inserted between the ribs in the ex vivo test the coating remains stably fixed on the chest tube. The stable fixation of the coating allows the surgeon to maintain their techniques for handling and placing the chest tube in the patient.

In addition, the stable fixation of the coating to the tube prevents obstruction of the chest tube openings or central lumen. Similarly, when using a Blake drain, none of the drain channels are blocked.

Coated Chest Tube is Able to Stably Release Lidocaine

A post-sterilization lidocaine release profile indicates continued stable elution over a period of about 3-7 days. The maximum subcutaneous dose of lidocaine hydrochloride is about 4.5 mg/kg with a maximum dose at one time not to exceed 300 mg.⁶⁷ The targeted lidocaine release for a chest tube or Blake drain is 4.5 mg/kg/day.

While this embodiment describes lidocaine as the anesthetic, other anesthetics are contemplated for use in the invention. Similarly, while a chest tube is used in this embodiment, other types of catheters may be similarly coated.

Phased Release with Lidocaine Loaded into Hydrogel and Micelles

By mixing the lidocaine with the gelatin, some lidocaine quickly releases out of the hydrogel while the lidocaine-loaded micelles embedded within the hydrogel can provide a slow, sustained release over time. The general procedure for loading the lidocaine into the hydrogel and micelles can remain the same as above. However, less than the initial amount of lidocaine may be loaded into the hydrogel as more lidocaine is added with the addition of the lidocaine-loaded micelles.

### CONCLUSION

The inventors have developed a novel nanoparticle capable of being loaded with a therapeutic agent such as an anesthetic, antimicrobial or growth factor. These nanoparticles can be embedded within a crosslinked hydrogel to form a drug delivery system. The drug delivery system can be administered directly to the patient or alternatively, are capable of being applied to various types of catheters or drains, such as chest tubes. Application of the composition to a catheter or drain allows for the sustained release of the therapeutic agent, such as an anesthetic, to the patient over an extended period of time.

### REFERENCES

(1) Mozaffarian, D.; Benjamin, E.; Go, A.; Arnett, D.; Blaha, M.; Cushman, M.; Das, S.; Ferranti, S.; Després, J.; Fullerton, H.; Howard, V.; Huffman, M.; Isasi, C.; Jiménez, M.; Judd, S.; Kissela, B.; Lichtman, J.; Lisabeth, L.; Liu, S.; Mackey, R.; Magid, D.; McGuire, D.; Mohler, E.; Moy, C.; Muntner, M.; Mussolino, M.; Nasir, K.; Neumar, R.; Nichol, G.; Palaniappan, L.; Pandey, D.; Reeves, M.; Rodriguez, C.; Rosamond, W.; Sorlie, P.; Stein, J.; Towfighi, A.; Turan, T.; Virani, S.; et al. Heart Disease and Stroke Statistics-2016 Update a Report from the American Heart Association. Circulation 2016, 133, e38-e48.
(2) Segers, V. F. M.; Lee, R. T. Protein Therapeutics for Cardiac Regeneration after Myocardial Infarction. J. Cardiovasc. Transl. Res. 2010, 3, 469-477.
(3) Hastings, C. L.; Roche, E. T.; Ruiz-hernandez, E.; Schenkelayland, K.; Walsh, C. J.; Duffy, G. P. Drug and Cell Delivery for Cardiac Regeneration. Adv. Drug Delivery Rev. 2015, 84, 85-106.
(4) Ruvinov, E.; Cohen, S. Alginate Biomaterial for the Treatment of Myocardial Infarction: Progress, Translational Strategies, and Clinical Outlook. From Ocean Algae to Patient Bedside. Adv. Drug Delivery Rev. 2016, 96, 54-76.
(5) Lee, D. J.; Rocker, A. J.; Bardill, J. R.; Shandas, R.; Park, D. A Sulfonated Reversible Thermal Gel for the Spatiotemporal Control of VEGF Delivery to Promote Therapeutic Angiogenesis. J. Biomed. Mater. Res., Part A 2018, 3053.
(6) Yun, D.; Lee, Y. M.; Laughter, M. R.; Freed, C. R.; Park, D. Substantial Differentiation of Human Neural Stem Cells into Motor Neurons on a Biomimetic Polyurea. Macromol. Biosci. 2015, 1206.
(7) Laughter, M. R.; Ammar, D. A.; Bardill, J. R.; Pena, B.; Kahook, M. Y.; Lee, D. J.; Park, D. A Self-Assembling Injectable Biomimetic Microenvironment Encourages Retinal Ganglion Cell Axon Extension in Vitro. ACS Appl. Mater. Interfaces 2016, 8, 20540-20548.
(8) Lee, D. J.; Fontaine, A.; Meng, X.; Park, D. Biomimetic Nerve Guidance Conduit Containing Intraluminal Microchannels with Aligned Nanofibers Markedly Facilitates in Nerve Regeneration. ACS Biomater. Sci. Eng. 2016, 2, 1403-1410.
(9) Laughter, M. R.; Bardill, J. R.; Ammar, D. A.; Pena, B.; Calkins, D. J.; Park, D. Injectable Neurotrophic Factor Delivery System Supporting Retinal Ganglion Cell Survival and Regeneration Following Optic Nerve Crush. ACS Biomater. Sci. Eng. 2018, 3374.
(10) Peña, B.; Martinelli, V.; Jeong, M.; Bosi, S.; Lapasin, R.; Taylor, M. R. G.; Long, C. S.; Shandas, R.; Park, D.; Mestroni, L. Biomimetic Polymers for Cardiac Tissue Engineering. Biomacromolecules 2016, 17, 1593-1601.
(11) Peña, B.; Bosi, S.; Aguado, B. A.; Borin, D.; Farnsworth, N. L.; Dobrinskikh, E.; Rowland, T. J.; Martinelli, V.; Jeong, M.; Taylor, M. R. G.; Long, C.; Shandas, R.; Sbaizero, O.; Prato, M.; Anseth, K.; Park, D.; Mestroni, L. Injectable Carbon Nanotube-Functionalized Reverse Thermal Gel Promotes Cardiomyocytes Survival and Maturation. ACS Appl. Mater. Interfaces 2017, 9, 31645-31656.
(12) Lee, D. J.; Cavasin, M. A.; Rocker, A. J.; Soranno, D. E.; Meng, X.; Shandas, R.; Park, D. An Injectable Sulfonated Reversible Thermal Gel for Therapeutic Angiogenesis to Protect Cardiac Function after a Myocardial Infarction. J. Biol. Eng. 2019, 13, No. 6.
(13) Sun, Q.; Silva, E.; Wang, A.; Fritton, J. C.; Mooney, D. J.; Schaffler, M. B.; Grossman, P. M.; Rajagopalan, S. Sustained Release of Multiple Growth Factors from Injectable Polymeric System as a Novel Therapeutic Approach towards Angiogenesis. Pharm. Res. 2010, 27, 264-271.
(14) Awada, H. K.; Hwang, M. P.; Wang, Y. Biomaterials Towards Comprehensive Cardiac Repair and Regeneration after Myocardial Infarction: Aspects to Consider and Proteins to Deliver. Biomaterials 2016, 82, 94-112.
(15) Awada, H. K.; Johnson, N. R.; Wang, Y. Sequential Delivery of Angiogenic Growth Factors Improves Revascularization and Heart Function after Myocardial Infarction. J. Controlled Release 2015, 207, 7-17.
(16) Tengood, J. E.; Kovach, K. M.; Vescovi, P. E.; Russell, A. J.; Little, S. R. Sequential Delivery of Vascular Endothelial Growth Factor and Sphingosine 1-Phosphate for Angiogenesis. Biomaterials 2010, 31, 7805-7812.
(17) Brudno, Y.; Ennett-Shepard, A. B.; Chen, R. R.; Aizenberg, M.; Mooney, D. J. Enhancing Microvascular Formation and Vessel Maturation through Temporal Control over Multiple Pro-Angiogenic and pro-Maturation Factors. Biomaterials 2013, 34, 9201-9209.
(18) Betsholtz, C. Insight into the Physiological Functions of PDGF through Genetic Studies in Mice. Cytokine Growth Factor Rev. 2004, 215-228.
(19) Carmeliet, P.; Jain, R. K. Molecular Mechanisms and Clinical Applications of Angiogenesis. Nature 2011, 473, 298-307.
(20) Greenberg, J. I.; Shields, D. J.; Barillas, S. G.; Acevedo, L. M.; Murphy, E.; Huang, J.; Scheppke, L.; Stockmann, C.; Johnson, R. S.; Angle, N.; Cheresh, D. A Role for VEGF as a Negative Regulator of Pericyte Function and Vessel Maturation. Nature 2008, 456, 809-813.
(21) Frangogiannis, N. G. Regulation of the Inflammatory Response in Cardiac Repair. Circ. Res. 2012, 110, 159-173.
(22) Stumpf, C.; Seybold, K.; Petzi, S.; Wasmeier, G.; Raaz, D.; Yilmaz, A.; Anger, T.; Daniel, W. G.; Garlichs, C. D. Interleukin-10 Improves Left Ventricular Function in Rats with Heart Failure Subsequent to Myocardial Infarction. Eur. J. Heart Failure 2008, 10, 733-739.
(23) Moore, K. W.; de Waal Malefyt, R.; Coffman, R. L.; O'Garra, A. Interleukin-10 and the I Nterleukin-10 R Eceptor. Annu. Rev. Immunol. 2001, 19, 683-765.
(24) Krishnamurthy, P.; Rajasingh, J.; Lambers, E.; Qin, G.; Losordo, D. W.; Kishore, R. IL-10 Inhibits Inflammation and Attenuates Left Ventricular Remodeling after Myocardial Infarction via Activation of STAT-3 and Suppression of HuR. Circ. Res. 2009, 104, e9-e18.
(25) Huhn, R. D.; Radwanski, E.; Gallo, J.; Affrime, M. B.; Sabo, R.; Gonyo, G.; Monge, A.; Cutler, D. L. Pharmacodynamics of Subcutaneous Recombinant Human Interleukin-10 in Healthy Volunteers. Clin. Pharmacol. Ther. 1997, 62, 171-180.
(26) Salek-Ardakani, S.; Arrand, J. R.; Shaw, D.; Mackett, M. Heparin and Heparan Sulfate Bind Interleukin-10 and Modulate Its Activity. Blood 2000, 96, 1879-1888.
(27) Nah, D. Y.; Rhee, M. Y. The Inflammatory Response and Cardiac Repair after Myocardial Infarction. Korean Circ. J. 2009, 39, 393-398.
(28) Frangogiannis, N. The Inflammatory Response in Myocardial Infarction. Cardiovasc. Res. 2002, 53, 31-47.
(29) Frangogiannis, N. G. The Inflammatory Response in Myocardial Injury, Repair, and Remodelling. Nat. Rev. Cardiol. 2014, 11, 255-265.
(30) Yun, D.; Laughter, M. R.; Park, D. A Biomimetic Reverse Thermal Gel for 3-Dimensional Neural Tissue Engineering. Austin J. Biomed. Eng. 2014, 1, 1-5.
(31) Peña, B.; Shandas, R.; Park, D. A Heparin-Mimicking Reverse Thermal Gel for Controlled Delivery of Positively Charged Proteins. J. Biomed. Mater. Res., Part A 2015, 103, 2102-2108.
(32) Peña, B.; Shandas, R.; Park, D. A Heparin-Mimicking Reverse Thermal Gel for Controlled Delivery of Positively Charged Proteins. J. Biomed. Mater. Res., Part A 2015, 103, 2102-2108.
(33) Chu, H.; Gao, J.; Chen, C.-W.; Huard, J.; Wang, Y. Injectable Fibroblast Growth Factor-2 Coacervate for Persistent Angiogenesis. Proc. Natl. Acad. Sci. U.S.A. 2011, 108, 13444-13449.
(34) Famili, A.; Kahook, M. Y.; Park, D. A Combined Micelle and Poly(Serinol Hexamethylene Urea)-Co-Poly(N-Isopropylacrylamide) Reverse Thermal Gel as an Injectable Ocular Drug Delivery System. Macromol. Biosci. 2014, 1-11.
(35) Dust, J. M.; Fang, Z.-h.; Harris, J. M. Proton NMR Characterization of Poly(Ethylene Glycols) and Derivatives. Macromolecules 1990, 23, 3742-3746.
(36) Kinsella, L.; Chen, H.; Smith, J. A.; Rudland, P. S.; Fernig, D. G. Interactions of Putative Heparin-Binding Domains of Basic Fibroblast Growth Factor and Its Receptor, FGFR-1, with Heparin Using Synthetic Peptides. Glycoconjugate J. 1998, 15, 419-422.
(37) Freeman, I.; Kedem, A.; Cohen, S. The Effect of Sulfation of Alginate Hydrogels on the Specific Binding and Controlled Release of Heparin-Binding Proteins. Biomaterials 2008, 29, 3260-3268.
(38) Yoo, S. Y.; Kwon, S. M. Angiogenesis and Its Therapeutic Opportunities. Mediators Inflammation 2013, No. 127170.
(39) Ucuzian, A. A.; Gassman, A. A.; East, A. T.; Greisler, H. P. Molecular Mediators of Angiogenesis. J. Burn Care Res. 2010, 31, 158-175.
(40) Klagsbrun, M.; Sato, Y. Regulators of Angiogenesis. Annu. Rev. Physiol. 1991, 53, 217-239.
(41) Hao, X.; Silva, E.; Månsson-Broberg, A.; Grinnemo, K. H.; Siddiqui, A. J.; Dellgren, G.; Wardell, E.; Brodin, L. Å.; Mooney, D. J.; Sylvén, C. Angiogenic Effects of Sequential Release of VEGF-A165 and PDGF-BB with Alginate Hydrogels after Myocardial Infarction. Cardiovasc. Res. 2007, 75, 178-185.
(42) Deveza, L.; Choi, J.; Yang, F. Therapeutic Angiogenesis for Treating Cardiovascular Diseases. Theranostics 2012, 2, 801-814.
(43) Eppler, S. M.; Combs, D. L.; Henry, T. D.; Lopez, J. J.; Ellis, S. G.; Yi, J. H.; Annex, B. H.; McCluskey, E. R.; Zioncheck, T. F. A Target-Mediated Model to Describe the Pharmacokinetics and Hemodynamic Effects of Recombinant Human Vascular Endothelial Growth Factor in Humans. Clin. Pharmacol. Ther. 2002, 72, 20-32.
(44) Fujita, M.; Ishihara, M.; Shimizu, M.; Obara, K.; Nakamura, S.; Kanatani, Y.; Morimoto, Y.; Takase, B.; Matsui, T.; Kikuchi, M.; Maehara, T. Therapeutic Angiogenesis Induced by Controlled Release of Fibroblast Growth Factor-2 from Injectable Chitosan/Non-Anticoagulant Heparin Hydrogel in a Rat Hindlimb Ischemia Model. Wound Repair Regener. 2007, 15, 58-65.
(45) Kim, M. S.; Bhang, S.-H.; Yang, H. S.; Rim, N. G.; Jun, I.; Kim, S. I.; Kim, B.-S.; Shin, H. Development of Functional Fibrous Matrices for the Controlled Release of Basic Fibroblast Growth Factor to Improve Therapeutic Angiogenesis. Tissue Eng., Part A 2010, 16, 2999-3010.
(46) Lee, K. W.; Yoon, J. J.; Lee, J. H.; Kim, S. Y.; Jung, H. J.; Kim, S. J.; Joh, J. W.; Lee, H. H.; Lee, D. S.; Lee, S. K. Sustained Release of Vascular Endothelial Growth Factor from Calcium-Induced Alginate Hydrogels Reinforced by Heparin and Chitosan. Transplant. Proc. 2004, 36, 2464-2465.
(47) Nie, T.; Baldwin, A.; Yamaguchi, N.; Kiick, K. L. Production of Heparin-Functionalized Hydrogels for the Development of Responsive and Controlled Growth Factor Delivery Systems. J. Controlled Release 2007, 122, 287-296.
(48) Freeman, I.; Cohen, S. The Influence of the Sequential Delivery of Angiogenic Factors from Affinity-Binding Alginate Scaffolds on Vascularization. Biomaterials 2009, 30, 2122-2131.
(49) Chen, R. R.; Silva, E.; Yuen, W. W.; Mooney, D. J. Spatio-Temporal VEGF and PDGF Delivery Patterns Blood Vessel Formation and Maturation. Pharm. Res. 2007, 24, 258-264.
(50) Ruvinov, E.; Leor, J.; Cohen, S. The Promotion of Myocardial Repair by the Sequential Delivery of IGF-1 and HGF from an Injectable Alginate Biomaterial in a Model of Acute Myocardial Infarction. Biomaterials 2011, 32, 565-578.
(51) Awada, H. K.; Long, D. W.; Wang, Z.; Hwang, M. P.; Kim, K.; Wang, Y. Biomaterials A Single Injection of Protein-Loaded Coacervate-Gel Significantly Improves Cardiac Function Post Infarction. Biomaterials 2017, 125, 65-80.
(52) Chapanian, R.; Amsden, B. G. Combined and Sequential Delivery of Bioactive VEGF165 and HGF from Poly(Trimethylene Carbonate) Based Photo-Cross-Linked Elastomers. J. Controlled Release 2010, 143, 53-63.
(53) Benzakour, O.; Merzak, A.; Dooghe, Y.; Pironin, M.; Lawrence, D.; Vigier, P. Transforming Growth Factor Beta Stimulates Mitogenically Mouse NIH3t3 Fibroblasts and Those Cells Transformed by the EJ-h-Ras Oncogene. Growth Factors 1992, 265.
(54) Klouda, L.; Mikos, A. G. Thermoresponsive Hydrogels in Biomedical Applications. Eur. J. Pharm. Biopharm. 2008, 68, 34-45.
(55) Phillips D M. JCAHO pain management standards are unveiled. Joint Commission on Accreditation of Healthcare Organizations. JAMA 2000; 284:428-9.
(56) Gawande A A. It's time to adopt electronic prescriptions for opioids. Ann Surg 2017; 265:693-4.
(57) U.S. Department of Health and Human Services (HHS), Office of the Surgeon General, Facing Addiction in America: The Surgeon General's Spotlight on Opioids. Washington, D.C.: HHS, September 2018.
(58) Cauley C E, Anderson G, Haynes A B, Menendez M, Bateman B T, Ladha K. Predictors of in-hospital postoperative opioid overdose after major elective operations: a nationally representative cohort study. Ann Surg 2017; 265:702-8.
(59) Alex J, Ansari J, Bahalkar P, Agarwala S, Rehman M U, Saleh A, Cowen M E. Comparison of the immediate postoperative outcome of using the conventional two drains versus a single drain after lobectomy. Ann Thorac Surg 2003; 76:1046-9.
(60) Ziyaeifard M, Azarfarin R, Golzari S E J. A review of current analgesic techniques in cardiac surgery. Is epidural worth it? J Cardiovasc Thorac Res 2014; 6:133-40.
(61) Demmy T L, Nwogu C, Solan P, Yendamuri S, Wilding G, DeLeon O. Chest tube delivered bupivacaine improves pain and decreases opioid use after thoracoscopy. Ann Thorac Surg 2009; 87:1040-7.
(62) Kang H, Chung Y S, Choe J W, Woo Y C, Kim S W, Park S J, Hong J. Application of lidocaine jelly on chest tubes to reduce pain caused by drainage catheter after coronary artery bypass surgery. J Korean Med Sci 2014; 29:1398-1403.
(63) Faller K E, McAndrew D J, Schneider J E, Lygate C A. Refinement of analgesia following thoracotomy and experimental myocardial infarction using the mouse grimace scale. Exp Physiol. 2015; 100:164-72.
(64) Deuis J R, Dvorakova L S, Vetter I. Methods used to evaluate pain behaviors in rodents. Front Mol Neurosci 2017; 10:284.
(65) Ljungqvist O, Scott M, Fearon K C. Enhanced recovery after surgery, a review. JAMA Surg 2017; 152:292-298.
(66) Institute for Laboratory Animal Research. 2011. Guide for the Care and Use of Laboratory Animals, 8th ed. Washington (DC): National Academies Press.
(67) APP Pharmaceuticals Lidocaine HCl package insert—Revised February 2010.
(68) Bigi A, Cojazzi G, Panzavolta S, Rubini K, Roveri N. Mechanical and thermal properties of gelatin films at different degrees of glutaraldehyde crosslinking. Biomaterials 2001; 22:763-8.
(69) BioGlue Surgical Adhesive, CryoLife. Retrieved Sep. 15, 2019.

The disclosures of all publications cited above are expressly incorporated herein by reference, each in its entirety, to the same extent as if each were incorporated by reference individually.

It is also to be understood that the following claims are intended to cover all of the generic and specific features of the invention herein described, and all statements of the scope of the invention which, as a matter of language, might be said to fall there between. Now that the invention has been described,

What is claimed is:

1. A system for delivery of a therapeutic agent comprising:
at least one nanoparticle wherein the at least one nanoparticle is a micelle formed of a polymer comprising a hydrophobic segment and at least one hydrophilic segment;
a therapeutic agent encapsulated within the at least one nanoparticle; and
a hydrogel wherein the at least one nanoparticle is embedded within the hydrogel;
wherein the system allows for sustained release of the at least one therapeutic agent over an extended period of time.

2. The system of claim 1, further comprising an amount of the therapeutic agent also embedded within the hydrogel to allow for immediate release of the therapeutic agent in addition to the sustained release of the therapeutic agent from the at least one nanoparticle.

3. The system of claim 1, wherein the hydrophobic segment is poly(serinol hexamethylene urea) (PSHU).

4. The system of claim 3, wherein the at least one hydrophilic segment is polyethylene glycol (PEG), polycaprolactone (PCL), poly(glycolic acid) (PGA), poly(lactic acid) (PLA), or poly(lactic acid-co-glycolic acid) (PLGA).

5. The system of claim 1, wherein the therapeutic agent is selected from anesthetics, antimicrobials, or growth factors.

6. The system of claim 5, wherein the therapeutic agent is an anesthetic selected from the group consisting of lidocaine, marcaine, bupivacaine, prilocaine, mepivacaine, etidocaine, ropivacaine, and levobupivacaine.

7. The system of claim 5, wherein the therapeutic agent is at least one growth factor selected from the group consisting of vascular endothelial growth factor (VEGF), platelet-derived growth factor (PDGF) and interleukin-10 (IL-10).

8. A method of reducing pain associated with placement of a catheter in a patient in need thereof comprising:
applying an anesthetic-eluting drug delivery system to a surface of the catheter to produce a coated catheter wherein the drug delivery system comprising
at least one nanoparticle wherein the at least one nanoparticle is a micelle formed of a polymer comprising a hydrophobic segment and at least one hydrophilic segment;
an anesthetic encapsulated within the at least one nanoparticle; and
a hydrogel wherein the at least one nanoparticle is embedded within the hydrogel; and
inserting the coated catheter into the patient;
wherein the system allows for sustained release of the anesthetic over an extended period of time to relieve the pain associated with the placement of the catheter.

9. The method of claim 8, wherein the drug delivery system further comprises an amount of the anesthetic also embedded within the hydrogel to allow for immediate release of the anesthetic in addition to the sustained release of the anesthetic from the at least one nanoparticle.

10. The method of claim 8, wherein the hydrophobic segment is poly(serinol hexamethylene urea) (PSHU) and the at least one hydrophilic segment is polyethylene glycol (PEG), polycaprolactone (PCL), poly(glycolic acid) (PGA), poly(lactic acid) (PLA), or poly(lactic acid-co-glycolic acid) (PLGA).

11. The method of claim 8, wherein the anesthetic is selected from the group consisting of lidocaine, marcaine, bupivacaine, prilocaine, mepivacaine, etidocaine, ropivacaine, and levobupivacaine.

12. The method of claim 8, wherein the hydrogel is gelatin crosslinked with glutaraldehyde.

13. A method of promoting angiogenesis in patient after myocardial infarction comprising:
administering a therapeutic agent delivery system to the patient, the therapeutic agent delivery system comprising
at least one nanoparticle;
a therapeutically effective amount of growth factors selected from vascular endothelial growth factor (VEGF), platelet-derived growth factor (PDGF), interleukin-10 (IL-10), or combinations thereof wherein at least one of the growth factors is at least partially encapsulated within the at least one nanoparticle; and
a hydrogel wherein the at least one nanoparticle is embedded within the hydrogel;
wherein the growth factors not encapsulated within the nanoparticle are embedded in the hydrogel;
wherein the delivery system allows for both immediate and sustained release of the combination of growth factors.

14. The method of claim 13, wherein the PDGF is at least partially encapsulated within the at least one nanoparticle.

15. The method of claim 14, wherein the combination of growth factors are released sequentially with the VEGF released first, the IL-10 released second and the PDGF released last.
